SEQUENCE LISTING (17 sequences)

SEQ ID NO: 1 - protein, 340 aa, Homo sapiens
MESRKDITNQEELWKMKPRRNLEEDDYLHKDTGETSMLKRPVLLHLHQTAHADEFDCPSELQHTQELFPQWHLPIKIAAIIASLTFLYTLLREVIHPLATSHQQYFYKIPILVINKVLPMVSITLLALVYLPGVIAAIVQLHNGTKYKKFPHWLDKWMLTRKQFGLLSFFFAVLHAIYSLSYPMRRSYRYKLLNWAYQQVQQNKEDAWIEHDVWRMEIYVSLGIVGLAILALLAVTSIPSVSDSLTWREFHYIQSKLGIVSLLLGTIHALIFAWNKWIDIKQFVWYTPPTFMIAVFLPIVVLIFKSILFLPCLRKKILKIRHGWEDVTKINKTEICSQLN

SEQ ID NO: 2 - protein, 340 aa, Mus sp.
MEISDDVTNPEQLWKMKPKGNLEDDSYSTKDSGETSMLKRPGLSHLQHAVHVDAFDCPSELQHTQEFFPNWRLPVKVAAIISSLTFLYTLLREIIYPLVTSREQYFYKIPILVINKVLPMVAITLLALVYLPGELAAVVQLRNGTKYKKFPPWLDRWMLAKKQFGLLSFFFAVLHAVYSLSYPMRRSYRYKLLNWAYKQVQQNKEDAWVEHDVWRMEIYVSLGIVGLAILALLAVTSIPSVSDSLTWREFHYIQSKLGIVSLLLGTVHALVFAWNKWVDVSQFVWYMPPTFMIAVFLPTLVLICKIALCLPCLRKKILKIRCGWEDVSKINRTEMASRLN

SEQ ID NO: 3 - protein, 340 aa, Macaca sp.
MESRKDITNEEELWKMKPRRNLEEDDYLHKDTGETSMLKRPVLLHLHQTAHADEFDCPSELQHTQELFPQWHLPIKIAAIIASLTFLYTLLREVIHPLATSHQQYFYKIPILVINKVLPMVSITLLALVYLPGVIAAIVQLHNGTKYKKFPHWLDKWMLTRKQFGLLSFFFAVLHAIYSLSYPMRRSYRYKLLNWAYQQVQQNKEDAWIEHDVWRMEIYVSLGIVGLAILALLAVTSIPSVSDSLTWREFHYIQSKLGIVSLLLGTIHALIFAWNKWIDIKQFVWYTPPTFMIAVFLPVVVLIFKSILFLPCLRKKILKIRHGWEDITKINKMEISSQLN

SEQ ID NO: 4 - protein, 108 aa, Mus sp.
DIQMTQSPSSLSASVGDRVTITCRASQSISNYLAWYQQKPGKAPKLLIYAASSLESGVPSRFSGSGSGTDFTLTISSLQPEDFATYYCQQYNSLPWTFGQGTKVEIKR

SEQ ID NO: 5 - protein, 114 aa, Mus sp.
DIVMSQSPSSLAVSVGEKVTMSCKSSQSLLYRSNQKNYLAWYQQKPGQSPKLLIYWASTRESGVPDRFTGSGSGTDFTLTISSVKAEDLAVYYCQQYYNYPRTFGGGTKVEIKR

SEQ ID NO: 6 - protein, 114 aa, Artificial Sequence
  Description of Artificial Sequence Synthetic polypeptide
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKR

SEQ ID NO: 7 - protein, 113 aa, Homo sapiens
EVQLVESGGGLVQPGGSLRLSCAASGFTFSSYAMSWVRQAPGKGLEWVSVISGDGGSTYYADSVKGRFTISRDNSKNTLYLQMNSLRAEDTAVYYCARGFDYWGQGTLVTVSS

SEQ ID NO: 8 - protein, 124 aa, Mus sp.
DVQVQESGPGLVKPSQSLSLTCTVTGYSITSDYAWNWIRQFPGNKLEWMGYISNSGSTSYNPSLKSRISITRDTSKNQFFLQLISVTTEDTATYYCARERNYDYDDYYYAMDYWGQGTTLTVSA

SEQ ID NO: 9 - protein, 124 aa, Artificial Sequence
  Description of Artificial Sequence Synthetic polypeptide
EVQLVESGGGLVQPGGSLRLSCAASGYSITSDYAWNWVRQAPGKGLEWVGYISNSGSTSYNPSLKSRFTISRDNSKNTLYLQMNSLRAEDTAVYYCARERNYDYDDYYYAMDYWGQGTLVTVSS

SEQ ID NO: 10 - protein, 124 aa, Artificial Sequence
  Description of Artificial Sequence Synthetic polypeptide
EVQLVESGGGLVQPGGSLRLSCAVSGYSITSDYAWNWVRQAPGKGLEWVGYISNSGSTSYNPSLKSRFTISRDNSKNTLYLQMNSLRAEDTAVYYCARERNYDYDDYYYAMDYWGQGTLVTVSS

SEQ ID NO: 11 - protein, 17 aa, Artificial Sequence
  Description of Artificial Sequence Synthetic peptide
KSSQSLLYRSNQKNYLA

SEQ ID NO: 12 - protein, 7 aa, Artificial Sequence
  Description of Artificial Sequence Synthetic peptide
WASTRES

SEQ ID NO: 13 - protein, 9 aa, Artificial Sequence
  Description of Artificial Sequence Synthetic peptide
QQYYNYPRT

SEQ ID NO: 14 - protein, 11 aa, Artificial Sequence
  Description of Artificial Sequence Synthetic peptide
GYSITSDYAWN

SEQ ID NO: 15 - protein, 17 aa, Artificial Sequence
  Description of Artificial Sequence Synthetic peptide
GYISNSGSTSYNPSLKS

SEQ ID NO: 16 - protein, 15 aa, Artificial Sequence
  Description of Artificial Sequence Synthetic peptide
ERNYDYDDYYYAMDY

SEQ ID NO: 17 - protein, 28 aa, Artificial Sequence
  Description of Artificial Sequence Synthetic peptide
NYDYDDYYYAMDYWGQGTLVTVSSCSTK

(19) United States
(12) Patent Application Publication
(10) Pub. No.: US 2016/0068607 A1
(43) Pub. Date: Mar. 10, 2016

(54) THERAPEUTIC PROTEIN FORMULATIONS

(71) Applicant: Genentech, Inc., South San Francisco, CA (US)

(72) Inventors: Bruce KABAKOFF, Pacifica, CA (US); Daren NELSON, San Francisco, CA (US); Jun OUYANG, Foster City, CA (US); Trevor Eli SWARTZ, San Carlos, CA (US)

(73) Assignee: GENENTECH, INC., South San Francisco, CA (US)

(21) Appl. No.: 14/948,073

(22) Filed: Nov. 20, 2015

Related U.S. Application Data
(63) Continuation of application No. 13/130,277, filed on Oct. 17, 2011, now abandoned.
(60) Provisional application No. 61/116,541, filed on Nov. 20, 2008.

(51) Int. Cl. C07K 16/30; A61K 39/395; A61K 47/26; A61K 47/48

(52) U.S. Cl. CPC C07K 16/3069; A61K 47/48638; A61K 47/48415; A61K 47/48384; A61K 39/39558; A61K 47/26; C07K 2317/94; A61K 2039/505

(57) ABSTRACT

The present invention generally concerns formulations having a pH that inhibits aspartyl isomerization at an Asp-Asp motif in a therapeutic protein contained in such a formulation.

### CROSS REFERENCE TO RELATED APPLICATION

This application claims the benefit of U.S. Provisional Application No. 61/116,541, filed Nov. 20, 2008, the disclosure of which is incorporated herein by reference in its entirety.

### FIELD OF THE INVENTION

The present invention generally concerns formulations having a pH that inhibits aspartyl isomerization at an Asp-Asp motif in a therapeutic protein contained in such a formulation.

### BACKGROUND OF THE INVENTION

### Protein Formulations

Advances in biotechnology have made it possible to produce a variety of proteins for pharmaceutical applications using recombinant DNA techniques. Because proteins are larger and more complex than traditional organic and inorganic drugs (i.e. possessing multiple functional groups in addition to complex three-dimensional structures), the formulation of such proteins poses special considerations. Proteins are susceptible to degradation, which can involve chemical instability (e.g., a modification of the protein by bond formation or cleavage resulting in a new chemical entity) or physical instability (e.g., changes in the higher order structure of the protein). Physical instability can result from denaturation, aggregation, precipitation or adsorption, for example. Chemical instability can result from deamidation, racemization, isomerization, hydrolysis, oxidation, beta elimination or disulfide exchange.

Formulations comprising slightly acidic buffers have been used for therapeutic proteins, including monoclonal antibodies, in order to minimize deamidation, aggregation, and fragmentation. See, e.g., U.S. Pat. No. 6,171,586 to Lam et al. (describing a stable aqueous antibody formulation comprising acetate buffer at pH 5.0); WO2004/019861 to Johnson et al. (describing a pegylated anti-TNFα Fab fragment formulated in acetate buffer at pH 5.5); WO2004/004639 to Nesta (describing huC242-DM1, a tumor-activated immunotoxin, formulated in a 50 mM succinic acid buffer at pH 6.0); WO03/039485 to Kaisheva et al. (reporting that Daclizumab, a humanized IL-2 receptor antibody, had the highest stability in sodium succinate buffer at pH 6.0); and WO03/015894 to Oliver et al. (describing an aqueous formulation of 100 mg/mL SYNAGIS® in histidine buffer at pH 6.0).

Under conditions of pH 4-6, aspartic acid (Asp) residues in a protein can degrade by undergoing isomerization. Asp isomerization proceeds through a cyclic imide intermediate (succinimide), which undergoes rapid hydrolytic cleavage to form isoaspartate (isoAsp) or Asp in a molar ratio of about 3:1. See Wakanar et al. Biochemistry 46:1534-1544 (2007). The residue on the C-terminal side of the Asp affects the susceptibility of the Asp to isomerization, with Asp that occurs in Asp-Gly being particularly susceptible to isomerization. Id. Asp isomerization in a therapeutic antibody can lead to substantial loss of antigen-binding activity, particularly if the Asp occurs in an antigen-binding region of the antibody, e.g., a complementarity determining region (CDR). Thus, there is a need in the art for formulations that inhibit aspartyl isomerization at an Asp-Asp motif in a therapeutic protein contained in such a formulation.

### Anti-STEAP-1 Antibodies

STEAP-1 is a cell surface antigen characterized by a molecular topology of six transmembrane domains and intracellular N- and C-termini, suggesting that it folds in a “serpentine” manner into three extracellular and two intracellular loops. STEAP-1 is expressed predominantly in prostate cells in normal human tissues. It is also expressed at high levels across various states of prostate cancer and in other human cancers, such as lung, colon, ovarian, bladder, and pancreatic cancer, and Ewing's sarcoma. See Hubert et al., Proc. Natl. Acad. Sci. USA 96:14523-14528 (1999); WO 99/62941; Challita-Eid et al. Cancer Res. 67:5798-5805; and WO2008/052187.)

Certain antibodies that bind to STEAP-1 have been described. (See WO2008/052187, which is expressly incorporated by reference herein.) Further, immunoconjugates derived from those antibodies have been shown to reduce tumor volume in prostate tumor xenograft models. Id. Thus, anti-STEAP-1 antibodies or immunoconjugates are useful for the treatment of cancer, e.g., prostate cancer. Accordingly, suitable formulations for administering anti-STEAP-1 antibodies or immunoconjugates would be useful in cancer treatment.

The inventions herein satisfy the above needs and provide further benefits.

### SUMMARY OF THE INVENTION

The invention herein relates, at least in part, to formulations that comprise a therapeutic protein having an Asp-Asp motif, wherein the formulation improves the stability of the protein by inhibiting aspartyl isomerization at an Asp residue in the Asp-Asp motif. In one aspect, the formulation has a pH that inhibits aspartyl isomerization of an Asp residue in the Asp-Asp motif.

In one aspect, a formulation comprising a therapeutic protein having an Asp-Asp motif is provided, wherein the pH of the formulation is greater than 6.0 and less than 9.0. In one embodiment, the pH is from 6.25 to 7.0. In another embodiment, the pH is about 6.5. In another embodiment, the therapeutic protein is an antibody. In one such embodiment, the antibody comprises a hypervariable region (HVR) that comprises an Asp-Asp motif. In one such embodiment, the Asp-Asp motif occurs in HVR-H3.

In a further embodiment, the antibody is an anti-STEAP-1 antibody that comprises an HVR-H3 comprising the amino acid sequence of SEQ ID NO:16. In one such embodiment, the anti-STEAP-1 antibody further comprises one or more HVRs selected from (a) an HVR-H1 comprising the amino acid sequence of SEQ ID NO:14; (b) an HVR-H2 comprising the amino acid sequence of SEQ ID NO:15; (c) an HVR-L1 comprising the amino acid sequence of SEQ ID NO:11; (d) an HVR-L2 comprising the amino acid sequence of SEQ ID NO:12; and (e) an HVR-L3 comprising the amino acid sequence of SEQ ID NO:13. In one such embodiment, the antibody comprises (a) an HVR-H1 comprising the amino acid sequence of SEQ ID NO:14; (b) an HVR-H2 comprising the amino acid sequence of SEQ ID NO:15; (c) an HVR-H3 comprising the amino acid sequence of SEQ ID NO:16; (d) an HVR-L1 comprising the amino acid sequence of SEQ ID NO:11; (e) an HVR-L2 comprising the amino acid sequence of SEQ ID NO:12; and (f) an HVR-L3 comprising the amino acid sequence of SEQ ID NO:13.

In a further embodiment, the antibody is an anti-STEAP-1 antibody that comprises an HVR-H3 comprising the amino acid sequence of SEQ ID NO:16 and that comprises a heavy chain variable region (VH) comprising an amino acid sequence having at least 90% amino acid sequence identity to an amino acid sequence selected from SEQ ID NOs:8-10. In one such embodiment, the antibody further comprises a light chain variable region (VL), wherein the VL comprises an amino acid sequence having at least 90% amino acid sequence identity to an amino acid sequence selected from SEQ ID NOs:5-6.

In a further embodiment, the antibody is conjugated to a cytotoxic agent. In one such embodiment, the cytotoxic agent is an auristatin. In another such embodiment, the cytotoxic agent is a maytansinoid drug moiety.

In a further embodiment, the antibody shows ≦25% loss of antigen binding when stored at 40° C. for four weeks, compared to storage at 5° C. for six months.

In a further embodiment, a formulation comprises a histidine-acetate buffer at a concentration of 20 mM. In a further embodiment, a formulation comprises a histidine-chloride buffer at a concentration of 20 mM. In a further embodiment, a formulation comprises a saccharide selected from trehalose and sucrose present in an amount from 60 mM to 250 mM. In a further embodiment, a formulation comprises polysorbate 20 in an amount from 0.01% to 0.1%.

Any of the above-described embodiments may be present singly or in combination.

In another aspect, a method of treating cancer is provided, the method comprising administering to a mammal a formulation comprising an anti-STEAP-1 antibody as in any of the embodiments provided above.

In a further aspect, a method of inhibiting aspartyl isomerization in a therapeutic protein comprising an Asp-Asp motif is provided, wherein the therapeutic protein is contained in a formulation, the method comprising raising the pH of the formulation to a pH sufficient to inhibit aspartyl isomerization. In one embodiment, the therapeutic protein is an antibody as in any of the embodiments provided above.

### BRIEF DESCRIPTION OF THE DRAWINGS

FIG. 1 shows an alignment of the amino acid sequences of STEAP-1 from human, mouse, and cynomolgus monkey.

FIGS. 2A and 2B shows the amino acid sequences of VL and VH domains, respectively, from certain anti-STEAP-1 antibodies.

FIG. 3 shows the elution profile resulting from ion exchange chromatography of an anti-STEAP-1 antibody formulation at pH 5.5 after various time periods of storage at 40° C., as described in Example A.

FIG. 4 shows a tryptic peptide map, indicating the presence of iso-Asp, as described in Example B.

FIG. 5 shows the results of electron transfer dissociation-mass spectrometry (ETD-MS), which identified the particular Asp residue undergoing isomerization, as described in Example B.

FIG. 6 shows that anti-STEAP-1 antibody formulations stored for 4 weeks at 40° C. showed loss of antigen binding. Formulations with increased pH showed decreased loss of binding at 40° C. No loss of binding was observed at any of the pHs tested when the formulations were stored at 5° C. for six months.

FIG. 7 shows the presence of antibody containing iso-Asp and succinimide after storage at 40° C. for various periods of time, as detected by hydrophobic interaction chromatography.

FIG. 8 shows the amount (expressed as a percentage) of Iso-Asp and succinimide in anti-STEAP-1 antibody preparations after storage at various temperatures for various periods of time, as described in Example D.

FIG. 9 assumes first order kinetics for the reaction of Asp to iso-Asp.

FIG. 10 shows the rates of Asp to iso-Asp isomerization determined at various temperatures, as described in Example E.

FIG. 11 shows an Arrhenius plot using the rates from FIG. 10. The plot predicts an activation energy of Asp-Asp isomerization to be about 25-30 Kcal/mol.

### DETAILED DESCRIPTION OF EMBODIMENTS

### I. Definitions

The term “formulation” refers to a preparation containing an active ingredient, and which contains no additional components which are unacceptably toxic to a subject to which the formulation would be administered. Such formulations are generally sterile.

A “sterile” formulation is aseptic or free from all living microorganisms and their spores.

Herein, a “frozen” formulation is one at a temperature below 0° C. Generally, the frozen formulation is not freeze-dried, nor is it subjected to prior, or subsequent, lyophilization. Preferably, the frozen formulation comprises frozen drug substance for storage (e.g., in stainless steel tank, PETG bottle, and Bioprocess Container™ storage systems (Hyclone, Logan, Utah)) or frozen drug product (in final vial configuration).

A “stable” formulation refers to a formulation in which the protein therein essentially retains physical stability and/or chemical stability and/or biological activity upon storage. Preferably, the protein essentially retains physical and chemical stability, as well as biological activity upon storage. The storage period is generally selected based on the intended shelf-life of the formulation. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993), for example. Stability can be measured at a selected temperature for a selected time period. Preferably, the formulation is stable at about 40′C for at least about 2-4 weeks; and/or stable at about 5° C. and/or 15° C. for at least 3 months, preferably 1-2 years; and/or stable at about −20° C. for at least 3 months, preferably at least 1-2 years. Furthermore, the formulation is preferably stable following freezing (to, e.g., −70° C.) and thawing of the formulation, for example following 1, 2 or 3 cycles of freezing and thawing. Stability can be evaluated qualitatively and/or quantitatively in a variety of different ways, including evaluation of aggregate formation (for example using size exclusion chromatography, by measuring turbidity, and/or by visual inspection); by assessing charge heterogeneity using cation exchange chromatography or capillary zone electrophoresis; amino-terminal or carboxy-terminal sequence analysis; mass spectrometric analysis; SDS-PAGE analysis to compare reduced and intact antibody; peptide map (for example tryptic or Lys-C) analysis; evaluating biological activity or antigen binding function of the antibody; etc. Instability may involve any one or more of: aggregation, deamidation (e.g. Asn deamidation), oxidation (e.g. Met oxidation), isomerization (e.g. Asp isomeriation), clipping/hydrolysis/fragmentation (e.g. hinge region fragmentation), succinimide formation, unpaired cysteine(s), N-terminal extension, C-terminal processing, glycosylation differences, etc. A formulation with “improved stability” means that a protein contained in the formulation retains greater physical stability and/or chemical stability and/or biological activity upon storage relative to the protein in a different formulation.

“Aspartyl isomerization” refers to conversion of an Asp residue in a protein to isoaspartic acid.

An “Asp-Asp” or “DD” motif refers to two consecutive aspartic acid residues in a protein.

“Inhibiting aspartyl isomerization,” and grammatical variants thereof, means that aspartyl isomerization at Asp-Asp is partially or completely inhibited in a protein contained in a given formulation at a given pH (e.g., 6.5) relative to the level of aspartyl isomerization at Asp-Asp in the protein contained in the same formulation at a lower pH (e.g., 5.5). Inhibition of aspartyl isomerization may be determined directly, e.g., by using HIC to quantify iso-Asp, or indirectly, e.g., by quantifying the biological activity of the protein. In one embodiment, aspartyl isomerization at Asp-Asp is inhibited by at least 10%, 20%, 30%, 40%, 50%, 60%, 70%, 80%, 90%, 95%, 96% 97%, 98%, 99% or 100%.

A “therapeutic protein” is a protein used in the treatment of a mammal having a disease or pathological condition. Therapeutic antibodies disclosed herein include anti-STEAP-1 antibodies.

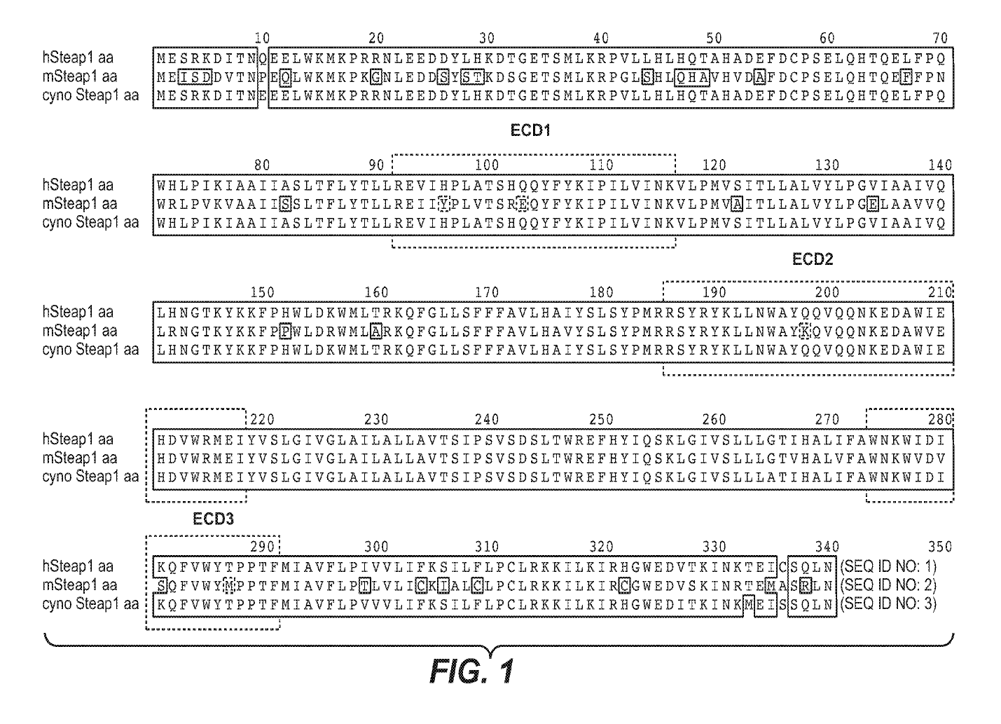

The term “STEAP-1” refers to any native STEAP-1 from any vertebrate source, including mammals such as primates (e.g. humans and monkeys) and rodents (e.g., mice and rats), unless otherwise indicated. The term encompasses “full-length,” unprocessed STEAP-1 as well as any form of STEAP-1 that results from processing in the cell. The term also encompasses naturally occurring variants of STEAP-1, e.g., splice variants or allelic variants. Exemplary STEAP-1 from human, mouse, and cynomolgus monkey are shown in FIG. 1.

The “biological activity” of an antibody refers to the ability of the antibody to bind to antigen.

By “isotonic” is meant that the formulation of interest has essentially the same osmotic pressure as human blood. Isotonic formulations will generally have an osmotic pressure from about 250 to 350 mOsm. Isotonicity can be measured using a vapor pressure or ice-freezing type osmometer, for example.

As used herein, “buffer” refers to a buffered solution that resists changes in pH by the action of its acid-base conjugate components. Examples of such buffers include acetate, succinate, gluconate, histidine, citrate, glycylglycine and other organic acid buffers.

A “histidine buffer” is a buffer comprising histidine ions. Examples of histidine buffers include histidine chloride, histidine acetate, histidine phosphate, and histidine sulfate. A histidine acetate buffer may be prepared by titrating L-histidine (free base, solid) with acetic acid (liquid).

A “saccharide” herein comprises the general composition (CH2O)n and derivatives thereof, including monosaccharides, disaccharides, trisaccharides, polysaccharides, sugar alcohols, reducing sugars, nonreducing sugars, etc. Examples of saccharides herein include glucose, sucrose, trehalose, lactose, fructose, maltose, dextran, glycerin, dextran, erythritol, glycerol, arabitol, sylitol, sorbitol, mannitol, mellibiose, melezitose, raffinose, mannotriose, stachyose, maltose, lactulose, maltulose, glucitol, maltitol, lactitol, iso-maltulose, etc. A saccharide herein may be a nonreducing disaccharide, such as trehalose or sucrose.

A “surfactant” refers to a surface-active agent, preferably a nonionic surfactant. Examples of surfactants herein include polysorbate (for example, polysorbate 20 and polysorbate 80); poloxamer (e.g. poloxamer 188); Triton; sodium dodecyl sulfate (SDS); sodium laurel sulfate; sodium octyl glycoside; lauryl-, myristyl-, linolcyl-, or stearyl-sulfobetainc; lauryl-, myristyl-, linolcyl- or stearyl-sarcosine; linolcyl-, myristyl-, or cetyl-betaine; lauroamidopropyl-, cocamidopropyl-, linoleamidopropyl-, myristamidopropyl-, palmidopropyl-, or isostearamidopropyl-betaine (e.g. lauroamidopropyl); myristamidopropyl-, palmidopropyl-, or isostearamidopropyl-dimethylamine; sodium methyl cocoyl-, or disodium methyl oleyl-taurate; and the MONAQUAT™ series (Mona Industries, Inc., Paterson, N.J.); polyethyl glycol, polypropyl glycol, and copolymers of ethylene and propylene glycol (e.g. Pluronics, PF68 etc); etc.

The term “about,” with reference to a numerical value, refers to that numerical value plus or minus 5%.

The term “antibody” herein is used in the broadest sense and specifically covers full length monoclonal antibodies, polyclonal antibodies, multispecific antibodies (e.g. bispecific antibodies), and antibody fragments, so long as they exhibit the desired biological activity.

The term “monoclonal antibody” as used herein refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen. In addition to their specificity, the monoclonal antibodies are advantageous in that they are uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, Ape etc) and human constant region sequences.

“Antibody fragments” comprise a portion of a full length antibody comprising an antigen-binding region thereof. Examples of antibody fragments include Fab, Fab′, F(ab′)₂, and Fv fragments; diabodies; linear antibodies; single-chain antibody molecules; and multispecific antibodies formed from antibody fragment(s).

A “full length antibody” is one which comprises an antigen-binding variable region as well as a light chain constant domain (C_{L}) and heavy chain constant domains, C_{H}1, C_{H}2 and C_{H}3. The constant domains may be native sequence constant domains (e.g. human native sequence constant domains) or amino acid sequence variants thereof. In certain embodiments, a full length antibody has one or more effector functions.

An “amino acid sequence variant” antibody herein is an antibody with an amino acid sequence which differs from a reference antibody. Ordinarily, amino acid sequence variants will possess at least about 70% homology with the reference antibody, and preferably, they will be at least about 80%, more preferably at least about 90% homologous with the reference antibody. The amino acid sequence variants possess substitutions, deletions, and/or additions at certain positions relative to the reference antibody. Examples of amino acid sequence variants herein include acidic variants (e.g. deamidated antibody variant), basic variants, antibody with an amino-terminal leader extension (e.g. VHS−) on one or two light chains thereof, antibody with a C-terminal lysine residue on one or two heavy chains thereof, etc, and includes combinations of variations to the amino acid sequences of heavy and/or light chains. In one embodiment, an antibody variant comprises an amino-terminal leader extension on one or two light chains thereof, optionally further comprising other amino acid sequence and/or glycosylation differences relative to the reference antibody.

A “glycosylation variant” antibody herein is an antibody with one or more carbohydrate moeities attached thereto which differ from one or more carbohydrate moieties attached to a reference antibody. Examples of glycosylation variants herein include antibody with a G1 or G2 oligosaccharide structure, instead of a G0 oligosaccharide structure, attached to an Fc region thereof, antibody with one or two carbohydrate moieties attached to one or two light chains thereof, antibody with no carbohydrate attached to one or two heavy chains of the antibody, etc, and combinations of glycosylation alterations.

An “amino-terminal leader extension” herein refers to one or more amino acid residues of the amino-terminal leader sequence that are present at the amino-terminus of any one or more heavy or light chains of an antibody. An exemplary amino-terminal leader extension comprises or consists of three amino acid residues, VHS, present on one or both light chains of an antibody variant.

“Homology” is defined as the percentage of residues in the amino acid sequence variant that are identical after aligning the sequences and introducing gaps, if necessary, to achieve the maximum percent homology. Methods and computer programs for the alignment are well known in the art. One such computer program is “Align 2”, authored by Genentech, Inc., which was filed with user documentation in the United States Copyright Office, Washington, D.C. 20559, on Dec. 10, 1991.

Antibody “effector functions” refer to those biological activities attributable to the Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; down regulation of cell surface receptors (e.g. B cell receptor; BCR), etc.

Depending on the amino acid sequence of the constant domain of their heavy chains, full length antibodies can be assigned to different “classes”. There are five major classes of full length antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

“Antibody-dependent cell-mediated cytotoxicity” and “ADCC” refer to a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells in summarized is Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or U.S. Pat. No. 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998).

“Human effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source thereof, e.g. from blood or PBMCs as described herein.

The term “Fc receptor” or “FcR” is used to describe a receptor that binds to the Fc region of an antibody. In one embodiment, an FcR is a native sequence human FcR. Moreover, a preferred FcR is one which binds an IgG antibody (a gamma receptor) and includes receptors of the FcγRI, FcγRII, and Fcγ RIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see review M. in Da\ddot{c}ron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

“Complement dependent cytotoxicity” or “CDC” refers to the ability of a molecule to lyse a target in the presence of complement. The complement activation pathway is initiated by the binding of the first component of the complement system (C1q) to a molecule (e.g. an antibody) complexed with a cognate antigen. To assess complement activation, a CDC assay, e.g. as described in Gazzano-Santoro et al., J. Immunol. Methods 202:163 (1996), may be performed.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end. The constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” or “HVR,” also called “complementarity determining region” or “CDR,” as used herein refers to the amino acid residues of an antibody which are primarily responsible for antigen-binding. There are generally three HVRs in the heavy chain (HVR-H1, HVR-H2, and HVR-H3), and three HVRs in the light chain (HVR-L1, HVR-L2, and HVR-L3). In some embodiments, the hypervariable region comprises amino acid residues 24-34 (HVR-L1), 50-56 (HVR-L2) and 89-97 (HVR-L3) in the light chain variable domain and 31-35 (HVR-H1), 50-65 (HVR-H2) and 95-102 (HVR-H3) in the heavy chain variable domain (Kabat et al., Sequences of Proteins of Immunological Interest, 5th Ed. Public Health Service, National Institutes of Health, Bethesda, Md. (1991)). HVR-H3 is believed to play a unique role in conferring fine specificity to antibodies. See, e.g., Xu et al. (2000) Immunity 13:37-45; Johnson and Wu (2003) in Methods in Molecular Biology 248:1-25 (Lo, ed., Human Press, Totowa, N.J.). “Framework Region” or “FR” residues are those variable domain residues other than the hypervariable region residues as herein defined.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments which have hinge cysteines between them. Other chemical couplings of antibody fragments are also known.

The “light chains” of antibodies from any vertebrate species can be assigned to one of two clearly distinct types, called kappa (κ) and lambda (λ), based on the amino acid sequences of their constant domains.

“Single-chain Fv” or “scFv” antibody fragments comprise the VH and VL domains of antibody, wherein these domains are present in a single polypeptide chain. Preferably, the Fv polypeptide further comprises a polypeptide linker between the VH and VL domains which enables the scFv to form the desired structure for antigen binding. For a review of scFv see Plückthun in The Pharmacology of Monoclonal Antibodies, vol. 113, Rosenburg and Moore eds., Springer-Verlag, New York, pp. 269-315 (1994).

The term “diabodics” refers to small antibody fragments with two antigen-binding sites, which fragments comprise a variable heavy domain (VH) connected to a variable light domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

“Humanized” forms of non-human (e.g., rodent) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human donor antibody, such as a synthetic antibody or a mouse, rat, rabbit or nonhuman primate antibody having the desired specificity, affinity, and/or capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

A “naked antibody” is an antibody (as herein defined) that is not conjugated to a heterologous molecule, such as a cytotoxic moiety or radiolabel.

An “affinity matured” antibody is one with one or more alterations in one or more hypervariable regions thereof which result in an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of CDR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

An “agonist antibody” is an antibody which binds to and activates a receptor. Generally, the receptor activation capability of the agonist antibody will be at least qualitatively similar (and may be essentially quantitatively similar) to a native agonist ligand of the receptor.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In certain embodiments, an antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or silver stain, or preferably by CE-SDS with fluorescent stain. Isolated antibody includes the antibody in situ within recombinant cells since at least one component of the antibody's natural environment will not be present. Ordinarily, however, isolated antibody will be prepared by at least one purification step.

A “growth inhibitory agent” when used herein refers to a compound or composition which inhibits growth of a cell, e.g., a STEAP-1-expressing cancer cell, either in vitro or in vivo. Thus, the growth inhibitory agent may be one which significantly reduces the percentage of STEAP-1-expressing cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topo II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (W B Saunders: Philadelphia, 1995), especially p. 13.

An antibody which “induces apoptosis” is one which induces programmed cell death as determined by binding of annexin V, fragmentation of DNA, cell shrinkage, dilation of endoplasmic reticulum, cell fragmentation, and/or formation of membrane vesicles (called apoptotic bodies). The cell is usually one which expresses the antigen (e.g., STEAP-1) to which the antibody binds. In one embodiment, the cell is a tumor cell. For example, phosphatidyl serine (PS) translocation can be measured by annexin binding; DNA fragmentation can be evaluated through DNA laddering; and nuclear/chromatin condensation along with DNA fragmentation can be evaluated by any increase in hypodiploid cells. In certain embodiment, an antibody which induces apoptosis is one which results in about 2 to 50 fold, preferably about 5 to 50 fold, and most preferably about 10 to 50 fold, induction of annexin binding relative to untreated cell in an annexin binding assay using cells that express an antigen to which the antibody binds.

“Treatment” refers to both therapeutic treatment and prophylactic or preventative measures. Those in need of treatment include those already with the disease as well as those in which the disease is to be prevented. Hence, the patient to be treated herein may have been diagnosed as having the disease or may be predisposed or susceptible to the disease.

The terms “cancer” and “cancerous” refer to or describe the physiological condition in mammals that is typically characterized by unregulated cell growth. Examples of cancer include, but are not limited to, carcinoma, lymphoma, blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophagael cancer, tumors of the biliary tract, as well as head and neck cancer. Specific examples of prostate cancer include androgen independent and androgen dependent prostate cancer.

The term “effective amount” refers to an amount of a drug effective to treat a disease in a patient. Where the disease is cancer, the effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may inhibit (partially or completely) growth and/or kill existing cancer cells, it may be cytostatic and/or cytotoxic. The effective amount may extend progression free survival, result in an objective response (including a partial response, PR, or complete response, CR), increase overall survival time, and/or improve one or more symptoms of cancer.

A “STEAP-1-expressing cancer” is one comprising cells which have STEAP-1 protein present at their cell surface. A STEAP-1 expressing cancer which “overexpresses” STEAP-1 is one which has significantly higher levels of STEAP-1 at the cell surface thereof; compared to a noncancerous cell of the same tissue type. Such overexpression may be caused by gene amplification or by increased transcription or translation. STEAP-1 expression (or overexpression) may be determined in a diagnostic or prognostic assay by evaluating levels of the STEAP-1 present on the surface of a cell (e.g. via an immunohistochemistry assay; IHC). Alternatively, or additionally, one may measure levels of STEAP-1-encoding nucleic acid in the cell, e.g. via fluorescent in situ hybridization (FISH; see WO98/45479 published October, 1998), southern blotting, or polymerase chain reaction (PCR) techniques, such as real time quantitative PCR (RT-PCR). One may also study STEAP-1 expression by measuring STEAP-1 present in a biological fluid such as serum, e.g., by detecting STEAP-1 present on the surface of circulating tumor cells (CTCs) (see, e.g., Schaffer et al., Clin. Cancer Res. 13:2023-2029 (2007). Aside from the above assays, various in vivo assays are available to the skilled practitioner. For example, one may expose cells within the body of the patient to an antibody which is optionally labeled directly or indirectly with a detectable label, e.g. a radioactive isotope, and binding of the antibody to cells in the patient can be evaluated, e.g. by external scanning for radioactivity or by analyzing a biopsy taken from a patient previously exposed to the antibody.

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents, and toxins such as small molecule toxins or enzymatically active toxins of bacterial, fungal, plant or animal origin, including fragments and/or variants thereof.

A “chemotherapeutic agent” is a chemical compound useful in the treatment of cancer. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calichcamicin gamma1I and calichcamicin omegaI1 (see, e.g., Agnew, Chem Intl. Ed. Engl., 33: 183-186 (1994)); dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; dacarbazine; mannomustine; mitobronitol; mitolactol; pipobroman; gacytosine; arabinoside (“Ara-C”); thiotepa; taxoid, e.g., paclitaxel (TAXOL®), albumin-engineered nanoparticle formulation of paclitaxel (ABRAXANE™), and docetaxel (TAXOTERE®); chloranbucil; 6-thioguanine; mercaptopurine; methotrexate; platinum agents such as cisplatin, oxaliplatin, and carboplatin; vincas, which prevent tubulin polymerization from forming microtubules, including vinblastine (VELBAN®), vincristine (ONCOVIN®), vindesine (ELDISINE®, FILDESIN®), and vinorelbine (NAVELBINE®); etoposide (VP-16); ifosfamide; mitoxantrone; leucovovin; novantrone; edatrexate; daunomycin; aminopterin; ibandronate; topoisomerase inhibitor RFS 2000; difluorometlhylornithine (DMFO); retinoids such as retinoic acid, including bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); BAY439006 (sorafenib; Bayer); SU-11248 (Pfizer); perifosine, COX-2 inhibitor (e.g. celecoxib or etoricoxib), proteosome inhibitor (e.g. PS341); bortezomib (VELCADE®); CCI-779; tipifarnib (R11577); orafenib, ABT510; Bcl-2 inhibitor such as oblimersen sodium (GENASENSE®); pixantrone; EGFR inhibitors (see definition below); tyrosine kinase inhibitors (see definition below); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovorin.

Also included in this definition are anti-hormonal agents that act to regulate or inhibit hormone action on tumors such as anti-estrogens with mixed agonist/antagonist profile, including, tamoxifen (NOLVADEX®), 4-hydroxytamoxifen, toremifene (FARESTON®), idoxifene, droloxifene, raloxifene (EVISTA®), trioxifene, keoxifene, and selective estrogen receptor modulators (SERMs) such as SERM3; pure anti-estrogens without agonist properties, such as fulvestrant (FASLODEX®), and EM800 (such agents may block estrogen receptor (ER) dimerization, inhibit DNA binding, increase ER turnover, and/or suppress ER levels); aromatase inhibitors, including steroidal aromatase inhibitors such as formestane and exemestane (AROMASIN®), and nonsteroidal aromatase inhibitors such as anastrazole (ARIMIDEX®), letrozole (FEMARA®) and aminoglutethimide, and other aromatase inhibitors including vorozole (RIVISOR®), megestrol acetate (MEGASE®), fadrozole, imidazole; lutenizing hormone-releasing hormone agonists, including leuprolide (LUPRON® and ELIGARD®), goserelin, buserelin, and tripterelin; sex steroids, including progestines such as megestrol acetate and medroxyprogesterone acetate, estrogens such as diethylstilbestrol and premarin, and androgens/retinoids such as fluoxymesterone, all transretionic acid and fenretinide; onapristone; anti-progesterones; estrogen receptor down-regulators (ERDs); anti-androgens such as flutamide, nilutamide and bicalutamide; testolactone; and pharmaceutically acceptable salts, acids or derivatives of any of the above; as well as combinations of two or more of the above.

### II. Antibodies and Immunoconjugates for Formulations

(A) Methods and Compositions

In one aspect, a therapeutic protein that can be formulated according to the present invention is a protein containing an Asp-Asp motif. In one embodiment, the therapeutic protein is an antibody or immunoconjugate. Such antibodies and immunoconjugates are exemplified as follows.

(i) Antigen Selection and Preparation

Preferably, the antigen to which an antibody binds is a protein and administration of the antibody to a mammal suffering from a disease or disorder can result in a therapeutic benefit in that mammal. However, antibodies directed against nonpolypeptide antigens (such as tumor-associated glycolipid antigens; see U.S. Pat. No. 5,091,178) are also contemplated.

Where the antigen is a polypeptide, it may be a transmembrane molecule (e.g. receptor) or ligand such as a growth factor. Exemplary antigens include molecules such as renin; a growth hormone, including human growth hormone and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor VIIIC, factor IX, tissue factor (TF), and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; RANTES (regulated on activation normally T-cell expressed and secreted); human macrophage inflammatory protein (MIP-1-alpha); a serum albumin such as human serum albumin; Muellerian-inhibiting substance; relaxin A-chain; relaxin B-chain; prorclaxin; mouse gonadotropin-associated peptide; a microbial protein, such as beta-lactamase; DNase; IgE; a cytotoxic T-lymphocyte associated antigen (CTLA), such as CTLA-4; inhibin; activin; vascular endothelial growth factor (VEGF); receptors for hormones or growth factors; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-b; platelet-derived growth factor (PDGF); fibroblast growth factor such as aFGF and bFGF; epidermal growth factor (EGF); transforming growth factor (TGF) such as TGF-alpha and TGF-beta, including TGF-b1, TGF-b2, TGF-b3, TGF-b4, or TGF-b5; a tumor necrosis factor (TNF) such as TNF-alpha or TNF-beta; insulin-like growth factor-I and -II (IGF-I and IGF-II); des(1-3)-IGF-I (brain IGF-I), insulin-like growth factor binding proteins; CD proteins such as CD3, CD4, CD8, CD19, CD20, CD22 and CD40; erythropoietin; osteoinductive factors; immunotoxins; a bone morphogenetic protein (BMP); an interferon such as interferon-alpha, -beta, and -gamma; colony stimulating factors (CSFs), e.g., M-CSF, GM-CSF, and G-CSF; interleukins (ILs), e.g., IL-1, IL-2, IL-3, IL-4, IL-5, IL-6, IL-7, IL-8, IL-9 and IL-10; superoxide dismutase; T-cell receptors; surface membrane proteins; decay accelerating factor; viral antigen such as, for example, a portion of the AIDS envelope; transport proteins; homing receptors; addressins; regulatory proteins; integrins such as CD11a, CD11b, CD11c, CD18, an ICAM, VLA-4 and VCAM; a tumor associated antigen such as HER2, HER3 or HER4 receptor; and fragments of any of the above-listed polypeptides.

Exemplary molecular targets for antibodies encompassed by the present invention include CD proteins such as CD3, CD4, CD8, CD19, CD20, CD22, CD34 and CD40; members of the ErbB receptor family such as the EGF receptor, HER2, HER3 or HER4 receptor; B cell surface antigens, such as CD20 or BR3; a member of the tumor necrosis receptor superfamily, including DR5; prostate cell surface antigens, e.g., Annexin 2, Cadherin-1, Cav-1, Cd34, CD44, EGFR, EphA2, ERGL, Fas, hepsin, HER2, KAI1, MSR1, PATE, PMEPA-1, Prostasin, Prostein, PSCA, PSGR, PSMA, RTVP-1, ST7, STEAP-1, STEAP-2, TMPRSS2, TRPM2, and Trp-p8; cell adhesion molecules such as LFA-1, Mac1, p150.95, VLA-4, ICAM-1, VCAM, alpha4/beta7 integrin, and alphav/beta3 integrin including either alpha or beta subunits thereof (e.g. anti-CD11a, anti-CD18 or anti-CD11b antibodies); growth factors such as VEGF as well as receptors therefor; tissue factor (TF); a tumor necrosis factor (TNF) such as TNF-alpha or TNF-beta, alpha interferon (alpha-IFN); an interleukin, such as IL-8; IgE; blood group antigens; flk2/flt3 receptor; obesity (OB) receptor; mp1 receptor; CTLA-4; protein C etc.

Soluble antigens or fragments thereof, optionally conjugated to other molecules, can be used as immunogens for generating antibodies. For transmembrane molecules, such as receptors, fragments of these (e.g. an extracellular domain of a receptor) can be used as the immunogen. Alternatively, cells expressing the transmembrane molecule can be used as the immunogen. Such cells can be derived from a natural source (e.g. cancer cell lines) or may be cells which have been transformed by recombinant techniques to express the transmembrane molecule. Other antigens and forms thereof useful for preparing antibodies will be apparent to those in the art.

For production of anti-STEAP-1 antibodies, a STEAP-1 antigen can be, e.g., a soluble form of STEAP-1, an extracellular loop of STEAP-1, or a portion thereof containing the desired epitope. Alternatively, cells expressing STEAP-1 at their cell surface (e.g. 293T cells transformed with a vector encoding STEAP-1 can be used to generate antibodies (see, e.g., Challita-Eid et al. Cancer Res. 67:5798-805 (2007)).

(ii) Monoclonal Antibodies

Monoclonal antibodies are obtained from a population of substantially homogeneous antibodies, i.e., the individual antibodies comprising the population are identical and/or bind the same epitope, except for possible variants that may arise during production of the monoclonal antibody. Thus, the modifier “monoclonal” indicates the character of the antibody as not being a mixture of discrete antibodies.

For example, the monoclonal antibodies may be made using the hybridoma method first described by Kohler et al., Nature, 256:495 (1975), or may be made by recombinant DNA methods (U.S. Pat. No. 4,816,567). In the hybridoma method, a mouse or other appropriate host animal, such as a hamster, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human mycloma and mouse-human heteromyeloma cell lines also have been described for the production of human monoclonal antibodies (Kozbor, J. Immunol., 133:3001 (1984); and Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA). The binding affinity of a monoclonal antibody can, for example, be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal. The monoclonal antibodies secreted by the subclones are suitably separated from the culture medium, ascites fluid, or serum by conventional antibody purification procedures such as, for example, protein A-Sepharose, hydroxylapatite chromatography, gel electrophoresis, dialysis, or affinity chromatography.

DNA encoding monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Plückthun, Immunol. Revs., 130:151-188 (1992).

In a further embodiment, monoclonal antibodies or antibody fragments can be isolated from antibody phage libraries generated using techniques described, e.g., in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Production of high affinity (nM range) human antibodies by chain shuffling (Marks et al., Bio/Technology, 10:779-783 (1992)), as well as combinatorial infection and in vivo recombination for constructing very large phage libraries (Waterhouse et al., Nuc. Acids. Res., 21:2265-2266 (1993)) is described. Thus, these techniques are viable alternatives to traditional monoclonal antibody hybridoma techniques for isolation of monoclonal antibodies.

The DNA also may be modified, for example, by substituting the coding sequence for human heavy chain and light chain constant domains in place of the homologous murine sequences (U.S. Pat. No. 4,816,567; and Morrison, et al., Proc. Natl Acad. Sci. USA, 81:6851 (1984)), or by covalently joining to the immunoglobulin coding sequence all or part of the coding sequence for a non-immunoglobulin polypeptide. Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

The amino acid sequence of monoclonal antibody heavy and light chains, or portions thereof, may be derived, e.g., from the corresponding DNA sequence. For example, the amino acid sequence of the VH, VL, and/or one or more HVRs may be ascertained.

(iii) Humanized Antibodies

Methods for humanizing non-human antibodies have been described in the art. Preferably, a humanized antibody has one or more amino acid residues introduced into it from a source which is non-human. These non-human amino acid residues are often referred to as “import” residues, which are typically taken from an “import” variable domain.

Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework region (FR) for the humanized antibody (Sims et al., J. Immunol., 151:2296 (1993); Chothia et al., J. Mol. Biol., 196:901 (1987)). Another method uses a particular framework region derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

In certain embodiments, antibodies are humanized with retention of high affinity for the antigen and other favorable biological properties. To achieve this goal, in one embodiment, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

A humanized antibody herein may, for example, comprise nonhuman hypervariable region residues incorporated into a human variable heavy domain and may further comprise a framework region (FR) substitution at a position selected from the group consisting of 69H, 71H and 73H utilizing the variable domain numbering system set forth in Kabat et al., Sequences of Proteins of Immunological Interest, 5th Ed. Public Health Service, National Institutes of Health, Bethesda, Md. (1991). In one embodiment, the humanized antibody comprises FR substitutions at two or all of positions 69H, 71H and 73H.

A humanized antibody of particular interest herein binds to STEAP-1 and contains an Asp-Asp motif WO 2008/052187 describes exemplary humanized anti-STEAP-1 antibodies having an Asp-Asp motif in HVR-H3. The amino acid sequences of the VH and VL of such antibodies, including the HVRs, are provided herein. All embodiments of such antibodies as described in WO 2008/052187 are expressly incorporated herein by reference.

The present application also contemplates affinity matured antibodies derived from any of the antibodies described herein, where such affinity matured antibodies preferably contain an Asp-Asp motif. The parent antibody may be a human antibody or a humanized antibody, as described herein. Various forms of humanized antibodies and affinity matured antibodies are contemplated. For example, the humanized antibody or affinity matured antibody may be an antibody fragment, such as a Fab, which is optionally combined with a constant region and/or conjugated with one or more cytotoxic agent(s) in order to generate an immunoconjugate. Alternatively, a humanized antibody or affinity matured antibody may be a full length antibody, such as a full length IgG1 antibody, which is optionally conjugated with one or more cytotoxic agent(s) in order to generate an immunoconjugate.

(iv) Human Antibodies

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and U.S. Pat. Nos. 5,591,669, 5,589,369 and 5,545,807.

Alternatively, phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats; for their review see, e.g., Johnson, Kevin S. and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905. Fv variable domain sequences selected from human-derived phage display libraries can be combined with known human constant domain sequences as described above. As discussed above, human antibodies may also be generated by in vitro activated B cells (see U.S. Pat. Nos. 5,567,610 and 5,229,275).

(v) Antibody Fragments

Various techniques have been developed for the production of antibody fragments. Traditionally, these fragments were derived via proteolytic digestion of full length antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. For example, antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. The antibody fragment may also be a “linear antibody”, e.g., as described in U.S. Pat. No. 5,641,870 for example. Such linear antibody fragments may be monospecific or bispecific.

(vi) Bispecific Antibodies

Bispecific antibodies are antibodies that have binding specificities for at least two different epitopes. Exemplary bispecific antibodies may bind to two different epitopes of the STEAP-1 protein. Other such antibodies may combine a STEAP-1 binding site with binding site(s) for another prostate cell surface antigen, e.g., Annexin 2, Cadherin-1, Cav-1, Cd34, CD44, EGFR, EphA2, ERGL, Fas, pepsin, HER2, KAI1, MSR1, PATE, PMEPA-1, Prostasin, Prostein, PSCA, PSGR, PSMA, RTVP-1, ST7, STEAP-2, TMPRSS2, TRPM2, and Trp-p8. (See, e.g., Tricoli et al. Cancer Res. 10:3943-3953 (2004) for listing of prostate cell surface antigens.) Alternatively, a STEAP-1 arm may be combined with an arm which binds to a triggering molecule on a leukocyte such as a T-cell receptor molecule (e.g. CD2 or CD3), or Fc receptors for IgG (FcγR), such as FcγRI (CD64), FcγRII (CD32) and FcγRIII (CD16) so as to focus cellular defense mechanisms to the STEAP-1-expressing cell. Bispecific antibodies may also be used to localize cytotoxic agents to cells which express STEAP-1. These antibodies possess a STEAP-1-binding arm and an arm which binds the cytotoxic agent (e.g. saporin, anti-interferon-α, vinca alkaloid, ricin A chain, methotrexate or radioactive isotope hapten). Bispecific antibodies can be prepared as full length antibodies or antibody fragments (e.g. F(ab′)₂ bispecific antibodies).

Methods for making bispecific antibodies are known in the art. Traditional production of full length bispecific antibodies is based on the coexpression of two immunoglobulin heavy chain-light chain pairs, where the two chains have different specificities (Millstein et al., Nature, 305:537-539 (1983)). Similar procedures are disclosed in WO 93/08829, and in Traunecker et al., EMBO J., 10:3655-3659 (1991). According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in U.S. Pat. No. 5,731,168, the interface between a pair of antibody molecules can be engineered to maximize the percentage of heterodimers which are recovered from recombinant cell culture. The preferred interface comprises at least a part of the C_{H}3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine) This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein full length antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al., J. Exp. Med., 175: 217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the HER2 receptor and normal human T cells, as well as trigger the lytic activity of human cytotoxic lymphocytes against human breast tumor targets. Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy-chain variable domain (VH) connected to a light-chain variable domain (VL) by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tutt et al. J. Immunol. 147: 60 (1991).

(vii) Other Amino Acid Sequence Modifications

Amino acid sequence modification(s) of the antibodies described herein are contemplated. For example, it may be desirable to improve the binding affinity and/or other biological properties of the antibody. Amino acid sequence variants of an antibody can be prepared by introducing appropriate nucleotide changes into the nucleic acid encoding the antibody, or by peptide synthesis. Such modifications include, for example, deletions from, and/or insertions into and/or substitutions of residues within the amino acid sequences of the antibody. Any combination of deletion, insertion, and substitution is made to arrive at the final construct, provided that the final construct possesses the desired characteristics. The amino acid changes also may alter post-translational processing of the antibody, such as changing the number or position of glycosylation sites.

A useful method for identification of certain residues or regions of an antibody that are preferred locations for mutagenesis is called “alanine scanning mutagenesis” as described by Cunningham and Wells Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with antigen, e.g., STEAP-1 antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed antibody variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of an antibody molecule include the fusion to the N- or C-terminus of the antibody to an enzyme (e.g. for ADEPT) or a polypeptide which increases the scrum half-life of the antibody.

Another type of variant is an amino acid substitution variant. These variants have at least one amino acid residue in an antibody replaced by a different residue. The sites of greatest interest for substitutional mutagenesis include the hypervariable regions, but FR or Fc region alterations are also contemplated. Conservative substitutions are shown in Table 1 under the heading of “preferred substitutions.” Substitutions that change one or more biological properties (e.g., stability or efficacy) but do not alter other properties (e.g., antigen specificity) may be made. If preferred substitutions results in an antibody with desired properties, then more substantial changes, denominated “exemplary substitutions” in Table 1, or as further described below in reference to amino acid classes, may be introduced and the antibody screened for further improved properties.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; | Leu |
| | Phe; Norleucine | |
| Leu (L) | Norleucine; Ile; Val; | Ile |
| | Met; Ala; Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Scr (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; | Leu |
| | Ala; Norleucine | |

Substantial modifications in the biological properties of an antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Amino acids may be grouped according to similarities in the properties of their side chains (in A. L. Lehninger, in Biochemistry, second ed., pp. 73-75, Worth Publishers, New York (1975)):

(1) non-polar: Ala (A), Val (V), Leu (L), Ile (I), Pro (P), Phe (F), Trp (W), Met (M)

(2) uncharged polar: Gly (G), Ser (S), Thr (T), Cys (C), Tyr (Y), Asn (N), Gln (Q)

(3) acidic: Asp (D), Glu (E)

(4) basic: Lys (K), Arg (R), His(H)

Alternatively, naturally occurring residues may be divided into groups based on common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

Any cysteine residue not involved in maintaining the proper conformation of an antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

In one embodiment, a substitutional variant involves substituting one or more hypervariable region residues of a parent antibody. Generally, the resulting variant(s) selected for further development will have improved biological properties relative to the parent antibody from which they are generated. A convenient way for generating such substitutional variants involves affinity maturation using phage display. Briefly, several hypervariable region sites (e.g. 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and its antigen. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antibody alters the original glycosylation pattern of the antibody. By altering is meant deleting one or more carbohydrate moieties found in the antibody, and/or adding one or more glycosylation sites that are not present in the antibody.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to an antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Where the antibody comprises an Fc region, the carbohydrate attached thereto may be altered. For example, antibodies with a mature carbohydrate structure that lacks fucose attached to an Fc region of the antibody are described in US Pat Appl No US 2003/0157108 A1, Presta, L. See also US 2004/0093621 A1 (Kyowa Hakko Kogyo Co., Ltd). Antibodies with a bisecting N-acetylglucosamine (GlcNAc) in the carbohydrate attached to an Fc region of the antibody are referenced in WO03/011878, Jean-Mairet et al. and U.S. Pat. No. 6,602,684, Umana et al. Antibodies with at least one galactose residue in the oligosaccharide attached to an Fc region of the antibody are reported in WO97/30087, Patel et al. See, also, WO98/58964 (Raju, S.) and WO99/22764 (Raju, S.) concerning antibodies with altered carbohydrate attached to the Fc region thereof. Antibody compositions comprising main species antibody with such carbohydrate structures attached to the Fc region are contemplated herein.

Nucleic acid molecules encoding amino acid sequence variants of an antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody.

(viii) Cysteine Engineered Antibodies

In one aspect, the antibodies of the invention include cysteine engineered antibodies (also called ThioMAbs) in which one or more amino acids of a parent antibody are replaced with a free cysteine amino acid as disclosed in WO2006/034488 (herein incorporated by reference in its entirety). A cysteine engineered antibody comprises one or more free cysteine amino acids having a thiol reactivity value in the range of 0.6 to 1.0. A free cysteine amino acid is a cysteine residue which has been engineered into the parent antibody and is not part of a disulfide bridge. Cysteine engineered antibodies are useful for attachment of cytotoxic and/or imaging compounds at the site of the engineered cysteine through, for example, a maleimide or haloacetyl. The nucleophilic reactivity of the thiol functionality of a Cys residue to a maleimide group is about 1000 times higher compared to any other amino acid functionality in a protein, such as amino group of lysine residues or the N-terminal amino group. Thiol specific functionality in iodoacetyl and maleimide reagents may react with amine groups, but higher pH (>9.0) and longer reaction times are required (Garman, 1997, Non-Radioactive Labelling: A Practical Approach, Academic Press, London).

Cysteine engineered antibodies may be useful in the treatment of cancer and include antibodies specific for cell surface and transmembrane receptors, and tumor-associated antigens (TAA). Such antibodies may be used as naked antibodies (unconjugated to a drug or label moiety) or as antibody-drug conjugates (ADC), also called immunoconjugates. Cysteine engineered antibodies of the invention may be site-specifically and efficiently coupled with a thiol-reactive reagent. The thiol-reactive reagent may be a multifunctional linker reagent, a capture label reagent, a fluorophore reagent, or a drug-linker intermediate. The cysteine engineered antibody may be labeled with a detectable label, immobilized on a solid phase support and/or conjugated with a drug moiety. Thiol reactivity may be generalized to any antibody where substitution of amino acids with reactive cysteine amino acids may be made within the ranges in the light chain selected from amino acid ranges: L-10 to L-20; L-38 to L-48; L-105 to L-115; L-139 to L-149; L-163 to L-173; and within the ranges in the heavy chain selected from amino acid ranges: H-35 to H-45; H-83 to H-93; H-114 to H-127; and H-170 to H-184, and in the Fc region within the ranges selected from H-268 to H-291; H-319 to H-344; H-370 to H-380; and H-395 to H-405, where the numbering of amino acid positions begins at position 1 of the Kabat numbering system (Kabat et al. (1991) Sequences of Proteins of Immunological Interest, 5th Ed. Public Health Service, National Institutes of Health, Bethesda, Md.) and continues sequentially thereafter as disclosed in WO 2006/034488. In particular embodiments, substitution of an amino acid with cysteine may be made at A118 of the heavy chain (i.e., A118C) according to EU numbering, and/or at V205 of the light chain (i.e., V205C) according to Kabat numbering. Thiol reactivity may also be generalized to certain domains of an antibody, such as the light chain constant domain (CL) and heavy chain constant domains, CH1, CH2 and CH3. Cysteine replacements resulting in thiol reactivity values of 0.6 and higher may be made in the heavy chain constant domains α, δ, ε, γ, and μ of intact antibodies: IgA, IgD, IgE, IgG, and IgM, respectively, including the IgG subclasses: IgG1, IgG2, IgG3, IgG4, IgA, and IgA2. Such antibodies and their uses are disclosed in WO 2006/034488.

Cysteine engineered antibodies of the invention preferably retain to at least some extent the antigen binding capability of the parent antibody. Thus, cysteine engineered antibodies are capable of binding, preferably specifically, to antigens. Such antigens include, for example, tumor-associated antigens (TAA), cell surface receptor proteins and other cell surface molecules, transmembrane proteins, signalling proteins, cell survival regulatory factors, cell proliferation regulatory factors, molecules associated with (for e.g., known or suspected to contribute functionally to) tissue development or differentiation, lymphokines, cytokines, molecules involved in cell cycle regulation, molecules involved in vasculogenesis and molecules associated with (for e.g., known or suspected to contribute functionally to) angiogenesis.

An antibody of the invention may be conjugated to other thiol-reactive agents in which the reactive group is, for example, a maleimide, an iodoacetamide, a pyridyl disulfide, or other thiol-reactive conjugation partner (Haugland, 2003, Molecular Probes Handbook of Fluorescent Probes and Research Chemicals, Molecular Probes, Inc.; Brinkley, 1992, Bioconjugate Chem. 3:2; Garman, 1997, Non-Radioactive Labelling: A Practical Approach, Academic Press, London; Means (1990) Bioconjugate Chem. 1:2; Hermanson, G. in Bioconjugate Techniques (1996) Academic Press, San Diego, pp. 40-55, 643-671). The partner may be a cytotoxic agent (e.g. a toxin such as doxorubicin or pertussis toxin), a fluorophore such as a fluorescent dye like fluorescein or rhodamine, a chelating agent for an imaging or radiotherapeutic metal, a peptidyl or non-peptidyl label or detection tag, or a clearance-modifying agent such as various isomers of polyethylene glycol, a peptide that binds to a third component, or another carbohydrate or lipophilic agent.

(ix) Screening for Antibodies with the Desired Properties

Techniques for generating antibodies have been described above. One may further select antibodies with certain biological characteristics, as desired.

For example, an antibody that binds to STEAP-1 on the surface of a cell may be identified using immunohistochemistry, FACs, or other suitable techniques. An antibody that binds to STEAP-1 and that inhibits tumor growth in vivo may be identified using an assay as described in Challita-Eid et al. Cancer Res. 67:5798-5805 (2007). Briefly, SCID mice containing the patient-derived androgen-dependent prostate cancer xenograft LAPC-9AD or bladder cancer UM-UC-3 xenograft may be treated with anti-STEAP-1 antibody (or an immunoconjugate comprising such antibody), and tumor volume and/or PSA levels are measured to assess efficacy. An antibody that binds to STEAP-1 and that blocks STEAP-1-mediated intercellular communication may be identified using an assay as described in Challita-Eid, supra. Briefly, donor and acceptor PC3 cells are loaded with appropriate donor and accetor dyes and mixed to allow intercellular communication to occur as detected by a color change.

(x) Immunoconjugates

The invention also pertains to immunoconjugates comprising an antibody conjugated to a cytotoxic agent such as a chemotherapeutic agent, toxin (e.g. a small molecule toxin or an enzymatically active toxin of bacterial, fungal, plant or animal origin, including fragments and/or variants thereof), or a radioactive isotope (i.e., a radioconjugate).

Chemotherapeutic agents useful in the generation of such immunoconjugates have been described above. Conjugates of an antibody and one or more small molecule toxins, such as a calicheamicin, a maytansine (U.S. Pat. No. 5,208,020), a trichothene, and CC1065 are also contemplated herein.

In one embodiment of the invention, the antibody is conjugated to one or more maytansine molecules (e.g. about 1 to about 10 maytansine molecules per antibody molecule). Maytansine may, for example, be converted to May-SS-Me which may be reduced to May-SH3 and reacted with modified antibody (Chari et al. Cancer Research 52: 127-131 (1992)) to generate a maytansinoid-antibody immunoconjugate.

Another immunoconjugate comprises an antibody conjugated to one or more calichcamicin molecules. The calichcamicin family of antibiotics are capable of producing double-stranded DNA breaks at sub-picomolar concentrations. Structural analogues of calicheamicin which may be used include, but are not limited to, γ₁^{I}, α₂^{I}, α₃^{I}, N-acetyl-γ₁^{I}, PSAG and θ_{I}¹ (Hinman et al. Cancer Research 53: 3336-3342 (1993) and Lode et al. Cancer Research 58: 2925-2928 (1998)). See, also, U.S. Pat. Nos. 5,714,586; 5,712,374; 5,264,586; and 5,773,001 expressly incorporated herein by reference.

Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. See, for example, WO 93/21232 published Oct. 28, 1993.

The present invention further contemplates an immunoconjugate formed between an antibody and a compound with nucleolytic activity (e.g. a ribonuclease or a DNA endonuclease such as a deoxyribonuclease; DNase). The present invention further contemplates an immunoconjugate formed between an antibody and a radioactive isotope. A variety of radioactive isotopes are available for the production of radioconjugated antibodies. Examples include At²¹¹, I¹³¹, I¹²⁵, Y⁹⁰, Re¹⁸⁶, Re¹⁸⁸, Sm¹⁵³, Bi²¹², P³² and radioactive isotopes of Lu.

In yet another embodiment, an antibody may be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g. avidin) which is conjugated to a cytotoxic agent.

Conjugates of an antibody and cytotoxic agent may be made using a variety of bifunctional protein coupling agents such as N-succinimidyl-3-(2-pyridyldithiol) propionate (SPDP), succinimidyl-4-(N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al. Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of the cytotoxic drug in the cell. For example, an acid-labile linker, peptidase-sensitive dimethyl linker or disulfide-containing linker (Chari et al. Cancer Research 52: 127-131 (1992)) may be used.

Typically, peptide-based drug moieties can be prepared by forming a peptide bond between two or more amino acids and/or peptide fragments. Such peptide bonds can be prepared, for example, according to the liquid phase synthesis method (see E. Schröder and K. Lübke, “The Peptides”, volume 1, pp 76-136, 1965, Academic Press) that is well known in the field of peptide chemistry. The auristatin/dolastatin drug moieties may be prepared according to the methods of: U.S. Pat. No. 5,635,483; U.S. Pat. No. 5,780,588; Pettit et al (1989) J. Am. Chem. Soc. 111:5463-5465; Pettit et al (1998) Anti-Cancer Drug Design 13:243-277; Pettit, G. R., et al. Synthesis, 1996, 719-725; and Pettit et al (1996) J. Chem. Soc. Perkin Trans. 1 5:859-863. See also Doronina (2003) Nat Biotechnol 21(7):778-784; “Monomethylvaline Compounds Capable of Conjugation to Ligands”, US Patent Application Publication No. 2005-0238649 A1, hereby incorporated by reference in its entirety (disclosing, e.g., linkers and methods of preparing monomethylvaline compounds such as MMAE and MMAF conjugated to linkers).

### Maytansine and Maytansinoids

In some embodiments, the immunoconjugate comprises an antibody (full length or fragments) of the invention conjugated to one or more maytansinoid molecules.

Maytansinoids are mitototic inhibitors which act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533.

Maytansinoid drug moieties are attractive drug moieties in antibody drug conjugates because they are: (i) relatively accessible to prepare by fermentation or chemical modification, derivatization of fermentation products, (ii) amenable to derivatization with functional groups suitable for conjugation through the non-disulfide linkers to antibodies, (iii) stable in plasma, and (iv) effective against a variety of tumor cell lines.

Maytansine compounds suitable for use as maytansinoid drug moieties are well known in the art, and can be isolated from natural sources according to known methods, produced using genetic engineering techniques (see Yu et al (2002) PNAS 99:7968-7973), or maytansinol and maytansinol analogues prepared synthetically according to known methods.

Exemplary maytansinoid drug moieties include those having a modified aromatic ring, such as: C-19-dechloro (U.S. Pat. No. 4,256,746) (prepared by lithium aluminum hydride reduction of ansamytocin P2); C-20-hydroxy (or C-20-demethyl)+/−C-19-dechloro (U.S. Pat. Nos. 4,361,650 and 4,307,016) (prepared by demethylation using Streptomyces or Actinomyces or dechlorination using LAH); and C-20-demethoxy, C-20-acyloxy (—OCOR), +/−dechloro (U.S. Pat. No. 4,294,757) (prepared by acylation using acyl chlorides). and those having modifications at other positions

Exemplary maytansinoid drug moieties also include those having modifications such as: C-9-SH (U.S. Pat. No. 4,424,219) (prepared by the reaction of maytansinol with H₂S or P₂S₅); C-14-alkoxymethyl(demethoxy/CH₂ OR) (U.S. Pat. No. 4,331,598); C-14-hydroxymethyl or acyloxymethyl (CH₂OH or CH₂OAc) (U.S. Pat. No. 4,450,254) (prepared from Nocardia); C-15-hydroxy/acyloxy (U.S. Pat. No. 4,364,866) (prepared by the conversion of maytansinol by Streptomyces); C-15-methoxy (U.S. Pat. Nos. 4,313,946 and 4,315,929) (isolated from Trewia nudlflora); C-18-N-demethyl (U.S. Pat. Nos. 4,362,663 and 4,322,348) (prepared by the demethylation of maytansinol by Streptomyces); and 4,5-deoxy (U.S. Pat. No. 4,371,533) (prepared by the titanium trichloride/LAH reduction of maytansinol).

Exemplary embodiments of maytansinoid drug moieties include: DM1; DM3; and DM4, having the structures:

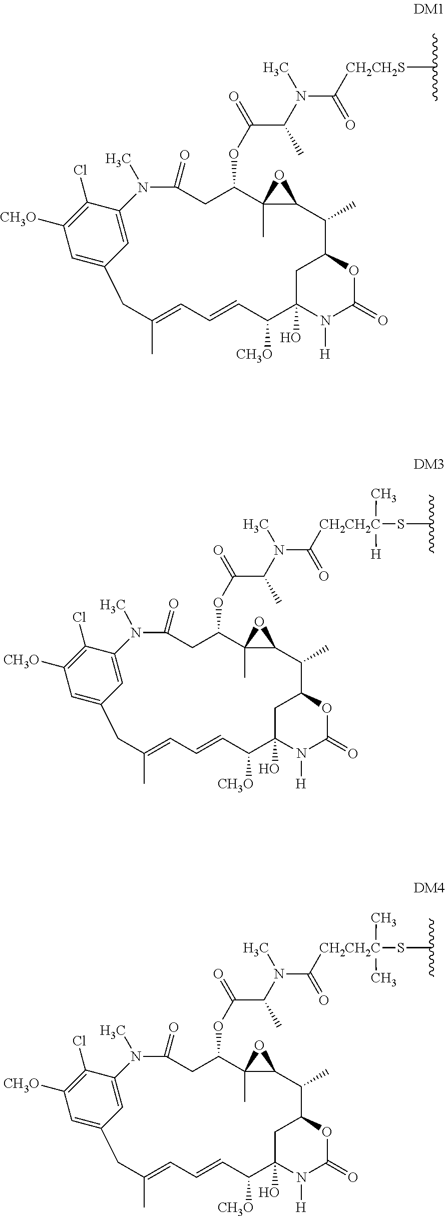

wherein the wavy line indicates the covalent attachment of the sulfur atom of the drug to a linker (L) of an antibody drug conjugate. HERCEPTIN® (trastuzumab) linked by SMCC to DM1 has been reported (WO 2005/037992, which is expressly incorporated herein by reference in its entirety). An antibody drug conjugate of the present invention may be prepared according to the procedures disclosed therein.

Other exemplary maytansinoid antibody drug conjugates have the following structures and abbreviations, (wherein Ab is antibody and p is 1 to about 8):

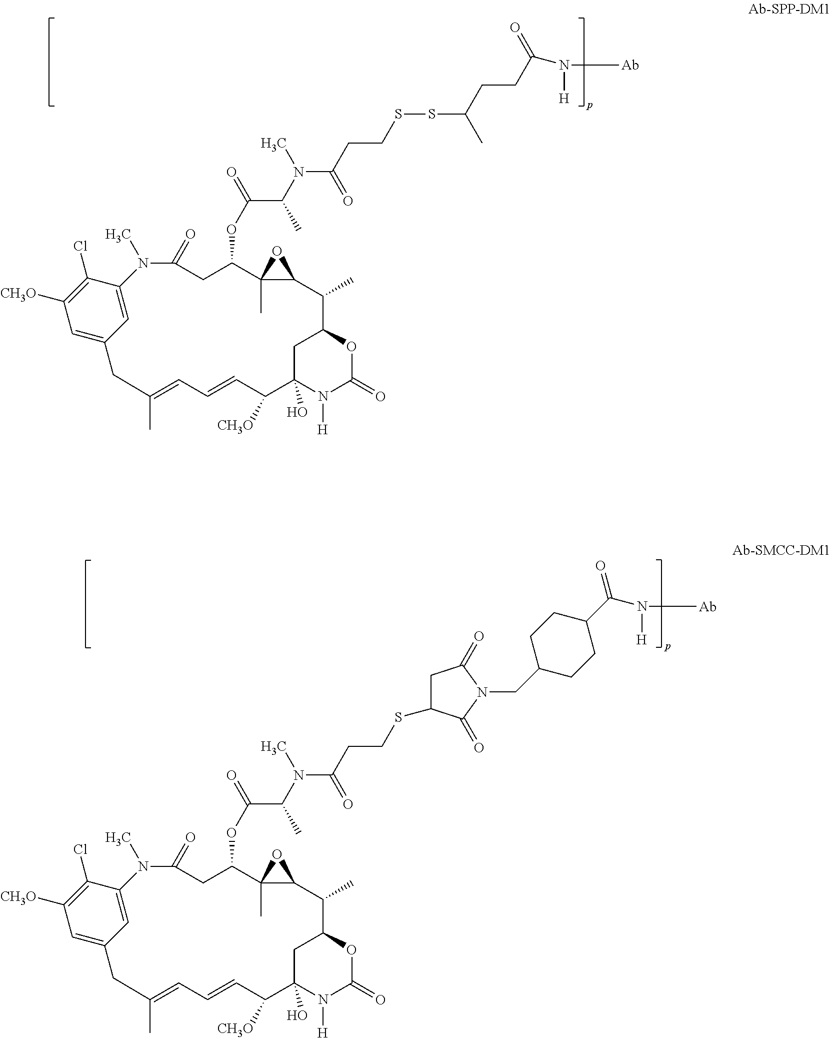

Exemplary antibody drug conjugates where DM1 is linked through a BMPEO linker to a thiol group of the antibody have the structure and abbreviation:

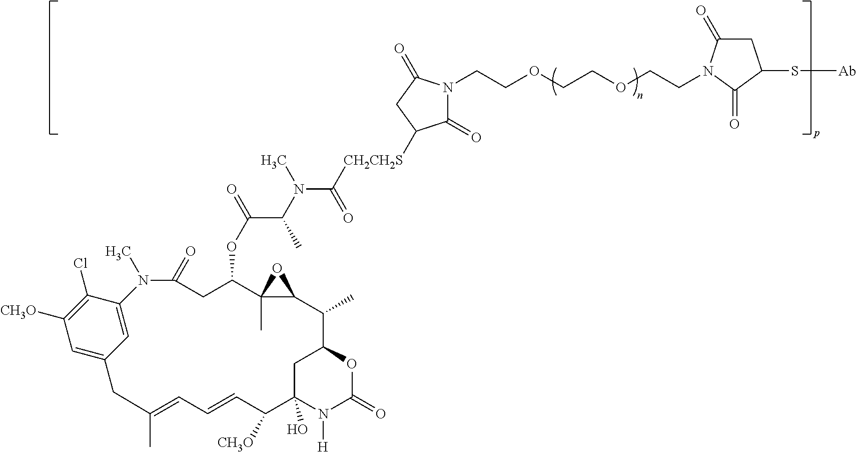

where Ab is antibody; n is 0, 1, or 2; and p is 1, 2, 3, or 4.

Immunoconjugates containing maytansinoids, methods of making same, and their therapeutic use are disclosed, for example, in U.S. Pat. Nos. 5,208,020; 5,416,064; 6,441,163 and European Patent EP 0 425 235 B1, the disclosures of which are hereby expressly incorporated by reference. Liu et al., Proc. Natl. Acad. Sci. USA 93:8618-8623 (1996) described immunoconjugates comprising a maytansinoid designated DM1 linked to the monoclonal antibody C242 directed against human colorectal cancer. The conjugate was found to be highly cytotoxic towards cultured colon cancer cells, and showed antitumor activity in an in vivo tumor growth assay. Chari et al., Cancer Research 52:127-131 (1992) describe immunoconjugates in which a maytansinoid was conjugated via a disulfide linker to the murine antibody A7 binding to an antigen on human colon cancer cell lines, or to another murine monoclonal antibody TA.1 that binds the HER-2/neu oncogene. The cytotoxicity of the TA.1-maytansonoid conjugate was tested in vitro on the human breast cancer cell line SK-BR-3, which expresses 3×10⁵ HER-2 surface antigens per cell. The drug conjugate achieved a degree of cytotoxicity similar to the free maytansinoid drug, which could be increased by increasing the number of maytansinoid molecules per antibody molecule. The A7-maytansinoid conjugate showed low systemic cytotoxicity in mice.

Anti-STEAP-1 antibody-maytansinoid conjugates are prepared by chemically linking an antibody to a maytansinoid molecule, preferably without significantly diminishing the biological activity of either the antibody or the maytansinoid molecule. See, e.g., U.S. Pat. No. 5,208,020 (the disclosure of which is hereby expressly incorporated by reference). An average of 3-4 maytansinoid molecules conjugated per antibody molecule has shown efficacy in enhancing cytotoxicity of target cells without negatively affecting the function or solubility of the antibody, although even one molecule of toxin/antibody would be expected to enhance cytotoxicity over the use of naked antibody. Maytansinoids are well known in the art and can be synthesized by known techniques or isolated from natural sources. Suitable maytansinoids are disclosed, for example, in U.S. Pat. No. 5,208,020 and in the other patents and nonpatent publications referred to hereinabove. Preferred maytansinoids are maytansinol and maytansinol analogues modified in the aromatic ring or at other positions of the maytansinol molecule, such as various maytansinol esters.

There are many linking groups known in the art for making antibody-maytansinoid conjugates, including, for example, those disclosed in U.S. Pat. Nos. 5,208,020, 6,441,163, or EP Patent 0 425 235 B1, Chari et al., Cancer Research 52:127-131 (1992), and US 2005/0169933 A1, the disclosures of which are hereby expressly incorporated by reference. Antibody-maytansinoid conjugates comprising the linker component SMCC may be prepared as disclosed in U.S. patent application Ser. No. 11/141,344, filed 31 May 2005, “Antibody Drug Conjugates and Methods”. The linking groups include disulfide groups, thioether groups, acid labile groups, photolabile groups, peptidase labile groups, or esterase labile groups, as disclosed in the above-identified patents. Additional linking groups are described and exemplified herein.

Conjugates of the antibody and maytansinoid may be made using a variety of bifunctional protein coupling agents (linkers) such as N-succinimidyl-3-(2-pyridyldithio) propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Preferred coupling agents include N-succinimidyl-3-(2-pyridyldithio) propionate (SPDP) (Carlsson et al., Biochem. J. 173:723-737 (1978)) and N-succinimidyl-4-(2-pyridylthio)pentanoate (SPP) to provide for a disulfide linkage.

The linker may be attached to the maytansinoid molecule at various positions, depending on the type of the link. For example, an ester linkage may be formed by reaction with a hydroxyl group using conventional coupling techniques. The reaction may occur at the C-3 position having a hydroxyl group, the C-14 position modified with hydroxymethyl, the C-15 position modified with a hydroxyl group, and the C-20 position having a hydroxyl group. In a preferred embodiment, the linkage is formed at the C-3 position of maytansinol or a maytansinol analogue.

In one embodiment, any of the antibodies of the invention (full length or fragment) is conjugated to one or more maytansinoid molecules. In one embodiment of the immunoconjugate, the cytotoxic agent D, is a maytansinoid DM1, DM3, or DM4. In one such embodiment of the immunoconjugate, the linker is selected from the group consisting of SPDP, SMCC, IT, SPDP, and SPP.

### Auristatin Immunoconjugates

In certain preferred embodiments, immunoconjugates comprise antibodies conjugated to dolastatins or dolostatin peptidic analogs and derivatives, the auristatins (U.S. Pat. Nos. 5,635,483; 5,780,588). Dolastatins and auristatins have been shown to interfere with microtubule dynamics, GTP hydrolysis, and nuclear and cellular division (Woyke et al (2001) Antimicrob. Agents and Chemother. 45(12):3580-3584) and have anticancer (U.S. Pat. No. 5,663,149) and antifungal activity (Pettit et al (1998) Antimicrob. Agents Chemother. 42:2961-2965). The dolastatin or auristatin drug moiety may be attached to the antibody through the N (amino) terminus or the C (carboxyl) terminus of the peptidic drug moiety (WO 02/088172).

Exemplary auristatin embodiments include the N-terminus linked monomethylauristatin drug moieties DE and DF, disclosed in “Monomethylvaline Compounds Capable of Conjugation to Ligands”, US Patent Application Publication No. 2005-0238649 A1, the disclosure of which is expressly incorporated by reference in its entirety. In further embodiments, monomethylauristatin drug moities include monomethyl auristatin E (MMAE) and monomethyl auristatin F (MMAF).

In further embodiments, an immunoconjugate having the formula Ab-(L-D)p is provided, wherein:

(a) Ab is an antibody,

(b) L is a linker;

(c) D is a drug of formula D_{F} or D_{F}

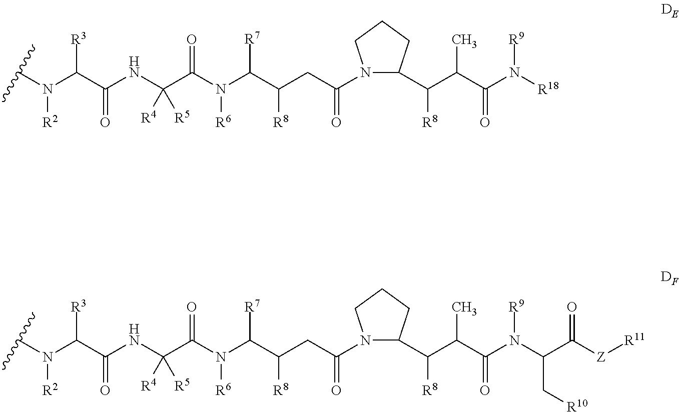

and wherein R² and R⁶ are each methyl, R³ and R⁴ are each isopropyl, R⁷ is sec-butyl, each R⁸ is independently selected from CH₃, O—CH₃, OH, and H; R⁹ is H; R¹⁰ is aryl; Z is —O— or —NH—; R¹¹ is H, C₁-C₈ alkyl, or —(CH₂)₂—O—(CH₂)₂—O—(CH₂)₂—O—CH₃; and R¹⁸ is —C(R⁸)₂—C(R⁸)₂-aryl; and

(d) p ranges from about 1 to 8.

Exemplary linker components (L) include the following, singly or in combination:
MC=6-maleimidocaproyl
Val-Cit or “vc”=valine-citrulline (an exemplary dipeptide in a protease cleavable linker)
Citrulline=2-amino-5-ureido pentanoic acid
PAB=p-aminobenzyloxycarbonyl (an example of a “self immolative” linker component)
Me-Val-Cit=N-methyl-valine-citrulline (wherein the linker peptide bond has been modified to prevent its cleavage by cathepsin B)
MC(PEG)6-OH=maleimidocaproyl-polyethylene glycol (can be attached to antibody cysteines).

In further embodiments, the linker is attached to the antibody through a thiol group on the antibody (e.g., a ThioMAb). In one embodiment, the linker is cleavable by a protease. In one embodiment, the linker comprises a val-cit dipeptide. In one embodiment, the linker comprises a p-aminobenzyl unit. In one embodiment, the p-aminobenzyl unit is disposed between the drug and a protease cleavage site in the linker. In one embodiment, the p-aminobenzyl unit is p-aminobenzyloxycarbonyl (PAB). In one embodiment, the linker comprises 6-maleimidocaproyl. In one embodiment, the 6-maleimidocaproyl is disposed between the antibody and a protease cleavage site in the linker. The above embodiments may occur singly or in any combination with one another.

In further embodiments, the drug is selected from the following:
MMAE=monomethyl auristatin E (MW 718)
MMAF=variant of auristatin E (MMAE) with a phenylalanine at the C-terminus of the drug (MW 731.5)
MMAF-DMAEA=MMAF with DMAEA (dimethylaminoethylamine) in an amide linkage to the C-terminal phenylalanine (MW 801.5)
MMAF-TEG=MMAF with tetraethylene glycol esterified to the phenylalanine
MMAF-NtBu=N-t-butyl, attached as an amide to C-terminus of MMAF
   In certain embodiments, the drug is selected from MMAE and MMAF.

In one embodiment, an immunoconjugate has the formula

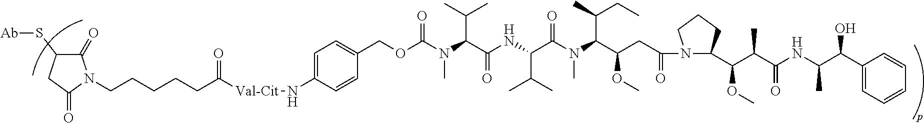

wherein Ab is an antibody, S is a sulfur atom, and p ranges from 2 to 5. In such embodiment, the immunoconjugate is designated Ab-MC-val-cit-PAB-MMAE. In another embodiment, an immunoconjugate is Ab-MC-MMAE.

In another embodiment, an immunoconjugate has the formula

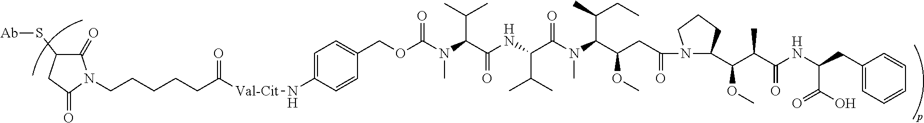

wherein Ab is an antibody, S is a sulfur atom, and p ranges from 2 to 5. In such embodiment, the immunoconjugate is designated Ab-MC-val-cit-PAB-MMAF. In another embodiment, an immunoconjugate is Ab-MC-MMAF.

(xi) Other Antibody Modifications

Other modifications of the antibody are contemplated herein. For example, the antibody may be linked to one of a variety of nonproteinaceous polymers, e.g., polyethylene glycol, polypropylene glycol, polyoxyalkylenes, or copolymers of polyethylene glycol and polypropylene glycol. The antibody also may be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980).

It may be desirable to modify the antibody of the invention with respect to effector function, e.g. so as to enhance antigen-dependent cell-mediated cyotoxicity (ADCC) and/or complement dependent cytotoxicity (CDC) of the antibody. This may be achieved by introducing one or more amino acid substitutions in an Fc region of the antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al. Cancer Research 53:2560-2565 (1993). Alternatively, an antibody can be engineered which has dual Fc regions and may thereby have enhanced complement lysis and ADCC capabilities. See Stevenson et al. Anti-Cancer Drug Design 3:219-230 (1989).

WO00/42072 (Presta, L.) describes antibodies with improved ADCC function in the presence of human effector cells, where the antibodies comprise amino acid substitutions in the Fc region thereof. Preferably, the antibody with improved ADCC comprises substitutions at positions 298, 333, and/or 334 of the Fc region. Preferably the altered Fc region is a human IgG1 Fc region comprising or consisting of substitutions at one, two or three of these positions.

Antibodies with altered C1q binding and/or complement dependent cytotoxicity (CDC) are described in WO99/51642, U.S. Pat. No. 6,194,551B1, U.S. Pat. No. 6,242,195B1, U.S. Pat. No. 6,528,624B1 and U.S. Pat. No. 6,538,124 (Idusogie et al.). The antibodies comprise an amino acid substitution at one or more of amino acid positions 270, 322, 326, 327, 329, 313, 333 and/or 334 of the Fc region thereof.

To increase the serum half life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule. Antibodies with substitutions in an Fc region thereof and increased serum half-lives are also described in WO00/42072 (Presta, L.).

Engineered antibodies with three or more (preferably four) functional antigen binding sites are also contemplated (US Appln No. US2002/0004587 A1, Miller et al.).

Antibodies disclosed herein may also be formulated as immunoliposomes. Liposomes containing the antibody are prepared by methods known in the art, such as described in Epstein et al., Proc. Natl. Acad. Sci. USA, 82:3688 (1985); Hwang et al., Proc. Natl Acad. Sci. USA, 77:4030 (1980); U.S. Pat. Nos. 4,485,045 and 4,544,545; and WO97/38731 published Oct. 23, 1997. Liposomes with enhanced circulation time are disclosed in U.S. Pat. No. 5,013,556.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al. J. Biol. Chem. 257: 286-288 (1982) via a disulfide interchange reaction. A chemotherapeutic agent is optionally contained within the liposome. See Gabizon et al. J. National Cancer Inst. 81(19)1484 (1989).

(B) Exemplary Antibodies and Immunoconjugates

Antibodies (e.g., monoclonal antibodies) containing an Asp-Asp motif are specifically contemplated for use with the formulations disclosed herein. For example, an antibody may contain an Asp-Asp motif in any region of a VH or VL. In specific embodiments, an Asp-Asp motif occurs in a region that influences antigen binding, including but not limited to any of the HVRs, and in certain embodiments, the HVR-H3.

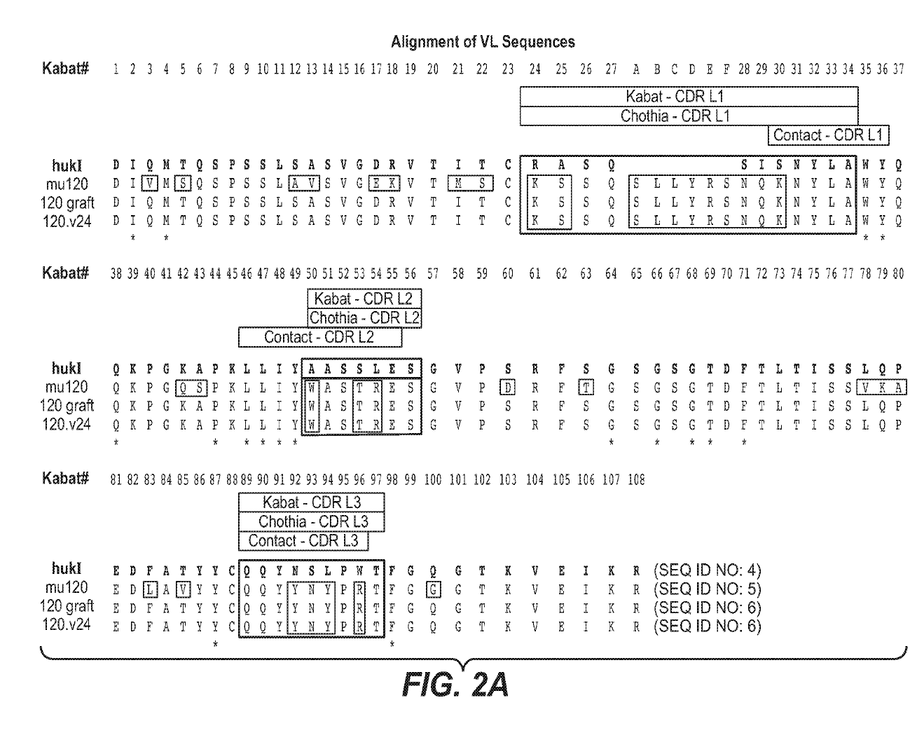

In one embodiment, an antibody containing an Asp-Asp motif is an anti-STEAP-1 antibody. WO 2008/052187 provides exemplary anti-STEAP-1 antibodies that contain an Asp-Asp motif in HVR-H3. All embodiments of such antibodies as described in WO 2008/052187 are expressly incorporated herein by reference. The amino acid sequences of the VH and VL of certain of those antibodies are provided herein in FIGS. 2A and 2B. The amino acid sequences of the HVRs of certain of those antibodies are provided below:

| | | |
|---|---|---|
| HVR-L1: | KSSQSLLYRSNQKNYLA | (SEQ ID NO: 11) |
| HVR-L2: | WASTRES | (SEQ ID NO: 12) |
| HVR-L3: | QQYYNYPRT | (SEQ ID NO: 13) |
| HVR-H1: | GYSITSDYAWN | (SEQ ID NO: 14) |
| HVR-H2: | GYISNSGSTSYNPSLKS | (SEQ ID NO: 15) |
| HVR-H3: | ERNYDYDDYYYAMDY | (SEQ ID NO: 16) |

Formulations comprising any of the antibodies described in WO 2008/052187 are expressly contemplated by the present invention.

In certain embodiments, an anti-STEAP-1 antibody comprises an Asp-Asp motif in a region that influences antigen binding, including but not limited to any of the HVRs, and in certain embodiments, the HVR-H3. In one embodiment, an anti-STEAP-1 antibody comprises an HVR-H3 comprising the amino acid sequence of SEQ ID NO:16. In one such embodiment, the anti-STEAP-1 antibody further comprises one or more HVRs selected from (a) an HVR-H1 comprising the amino acid sequence of SEQ ID NO:14; (b) an HVR-H2 comprising the amino acid sequence of SEQ ID NO:15; (c) an HVR-L1 comprising the amino acid sequence of SEQ ID NO:11; (d) an HVR-L2 comprising the amino acid sequence of SEQ ID NO:12; and (c) an HVR-L3 comprising the amino acid sequence of SEQ ID NO:13. In one such embodiment, the antibody comprises (a) an HVR-H1 comprising the amino acid sequence of SEQ ID NO:14; (b) an HVR-H2 comprising the amino acid sequence of SEQ ID NO:15; (c) an HVR-H3 comprising the amino acid sequence of SEQ ID NO:16; (d) an HVR-L1 comprising the amino acid sequence of SEQ ID NO:11; (e) an HVR-L2 comprising the amino acid sequence of SEQ ID NO:12; and (f) an HVR-L3 comprising the amino acid sequence of SEQ ID NO:13.

In certain embodiments, an anti-STEAP-1 antibody comprises a heavy chain variable region (VH), wherein the VH comprises an amino acid sequence having at least 90%, 91%, 92%, 93%, 94%, 95%, 96%, 97%, 98%, 99%, or 100% amino acid sequence identity to an amino acid sequence selected from SEQ ID NOs:8-10. In one embodiment, the antibody further comprises a light chain variable region (VL), wherein the VL comprises an amino acid sequence having at least 90%, 91%, 92%, 93%, 94%, 95%, 96%, 97%, 98%, 99%, or 100% amino acid sequence identity to an amino acid sequence selected from SEQ ID NOs:5-6. In any of the above embodiments, the Asp-Asp motif in HVR-H3 is conserved. In any of the above VH embodiments, the VH comprises an HVR-H3 comprising the amino acid sequence of SEQ ID NO:16 and optionally at least one HVR selected from (a) an HVR-H1 comprising the amino acid sequence of SEQ ID NO:14; and (b) an HVR-H2 comprising the amino acid sequence of SEQ ID NO:15. In any of the above VL embodiments, the VL comprises at least one, two, or three HVRs selected from (a) an HVR-L1 comprising the amino acid sequence of SEQ ID NO:11; (b) an HVR-L2 comprising the amino acid sequence of SEQ ID NO:12; and (c) an HVR-L3 comprising the amino acid sequence of SEQ ID NO:13. In certain embodiments, VH and VL are paired according to FIGS. 2A and 2B, e.g., SEQ ID NO:5 with SEQ ID NO:8 and SEQ ID NO:6 with SEQ ID NO:9 or 10.

Exemplary antibodies for use in any of the immunoconjugates described above is an anti-STEAP-1 antibody as described herein. Preferred anti-STEAP-1 antibodies and immunoconjugates (including ThioMAb immunoconjugates) are also described in WO 2008/052187, which is expressly incorporated by reference herein. Formulations comprising such immunoconjugates are expressly contemplated by the present invention. In certain embodiments, any of the above anti-STEAP-1 antibodies is conjugated to a cytotoxic agent. In one embodiment, the cytotoxic agent is an auristatin. In one such embodiment, the auristatin is MMAE or MMAF.

### III. Exemplary Formulations

The invention herein relates, at least in part, to formulations that comprise a therapeutic protein having an Asp-Asp motif, wherein the formulation has a pH that inhibits aspartyl isomerization of an Asp residue in the Asp-Asp motif.

In one aspect, a formulation is provided that comprises a therapeutic protein having an Asp-Asp motif, wherein the pH of the formulation is greater than 6.0 and less than 9.0. In one embodiment, the pH is greater than 6.0 and less than 8.0. In another embodiment, the pH is from 6.25 to 7.5. In another embodiment, the pH is from 6.25 to 7.0. In another embodiment, the pH is from 6.5 to 7.5. In another embodiment, the pH is from 6.5 to 7.0. In another embodiment, the pH is about 6.5. In another embodiment, the pH is within the range of 6.0-9.0, and the starting- and end-points of the range are selected from 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, and 9.0, with the starting-point being a lower pH than the end-point pH.

A particularly suitable pH, or pH range, for a particular therapeutic protein may be determined experimentally, e.g., by formulating a therapeutic protein containing an Asp-Asp motif at various pHs, and selecting a pH that optimizes the stability of the protein. For example, a pH that shows maximal inhibition of Asp-Asp isomerization (e.g., a basic pH) may lead to undesired levels of deamidation, aggregation, and fragmentation, whereas a pH that minimizes deamidation, aggregation, and fragmentation (e.g., an acidic pH) may lead to undesired levels of Asp-Asp isomerization. A pH that optimizes the stability of the protein may thus be achieved by balancing these degradative processes. Based on the teachings herein, such a pH is expected to fall within the ranges provided above, which include slightly acidic and basic pHs.

In certain embodiments, a method of inhibiting aspartyl isomerization in a therapeutic protein comprising an Asp-Asp motif is provided, wherein the therapeutic protein is contained in a formulation, the method comprising raising the pH of the formulation to a pH sufficient to inhibit aspartyl isomerization in the protein. Such pH may be any of those described above. Aspartyl isomerization may be inhibited by by at least 10%, 20%, 30%, 40%, 50%, 60%, 70%, 80%, 90%, 95%, 96% 97%, 98%, 99% or 100%, relative to that observed at the starting pH. In certain embodiments, a method of inhibiting aspartyl isomerization in a therapeutic protein comprising an Asp-Asp motif is provided, the method comprising maintaining the therapeutic protein in a formulation as provided in any of the above embodiments herein. In certain of the above embodiments, aspartyl isomerization is inhibited in the therapeutic protein where the pH of the formulation is 6.5 compared to the level of isomerization where the pH of the formulation is 5.5. The therapeutic protein can be an antibody, e.g., any of the anti-STEAP-1 antibodies provided herein, or ADCs thereof. The formulation can be a formulation as described herein.

Asp-Asp isomerization may be determined using various analytical methods, e.g., mass spectrometry, peptide mapping, electron transfer dissociation-mass spectrometry, and hydrophobic interaction chromatrography (HIC), as described in the examples herein.

Deamidation, aggregation and/or fragmentation of a therapeutic protein in a formulation may be determined by analytical methods such as those reviewed in Daugherty et al., Advanced Drug Deliver Reviews 58:686-706 (2006). Exemplary methods of assessing deamidation, aggregation and/or fragmentation are further provided below.

Aggregation can be assessed by observing the color, appearance, and clarity of the samples against a white and black background under white fluorescence light at room temperature. Additionally, UV absorbance of the formulation (diluted or not) can be used to assess aggregation. In one embodiment, UV absorbance is measured in a quartz cuvette with 1 cm path length on an HP 8453 spectrophotometer at 278 nm and 320 nm. The absorbance from 320 nm is used to correct background light scattering due to larger aggregates, bubbles and particles. The measurements are blanked against formulation buffer. Protein concentration is determined using the absorptivity of 1.65 (mg/mL)⁻¹cm⁻¹.

Cation exchange chromatography can be employed to measure changes in charge variants. In one embodiment, this assay utilizes a DIONEX PROPAC WCX-10™ column on an HP 1100™ HPLC system. Samples are diluted to 1 mg/mL with the mobile phase A containing 20 mM HEPES at pH 7.9. 30-50 μL of diluted samples are then loaded on the column kept at 40° C. Peaks are eluted with a shallow NaCl gradient using mobile B containing 20 mM HEPES, 200 mM NaCl, pH 7.9. The eluent is monitored at 280 nm. The data are analyzed using HP CHEMSTATION™ software (Rev B.01.03 or newer).

Purity of Fab and F(ab′)₂ fragments in a formulation can be determined by capillary zone electrophoresis (CZE). This assay can be run on a BIORAD BIOFOCUS™ 3000™ capillary electrophoresis system with a BIOCAP XL™ capillary, 50 um I.D., 44.6 cm total length and 40 cm to the detector.

Size exclusion chromatography can be used to quantitate aggregates and fragments. This assay can utilize a TSK G3000 SWXL™, 7.8×300 mm column on an HP 1100™ HPLC system. Samples are diluted to 1-2 mg/mL with the mobile phase and injection volume at 25-50 The mobile phase is 200 mM potassium phosphate and 250 mM potassium chloride at pH 6.2, and the protein is eluted with an isocratic gradient at 0.5 mL/min for 30 minutes. The eluent absorbance is monitored at 280 nm. Integration is done using HP CHEMSTATION™ software (Rev B.01.03 or newer).

Stability of a therapeutic protein in a formulation can also be assessed by determining the activity of the protein. Where the therapeutic protein is an antibody, stability can be assessed by determining whether and/or to what extent the antibody's ability to bind antigen is maintained, e.g. by ELISA or by a cell-based assay in the case of a cell surface antigen, such as the cell-based assay described in Example A herein. In certain embodiments, antibody in the formulation (such as any of the anti-STEAP-1 antibodies or immunoconjugates provided herein) shows ≦40% or 30%, and preferably ≦25%, 20%, 19%, 18%, 17%, 16%, 15%, 14%, 13%, 12%, 11%, 10%, 9%, 8%, 7%, 6%, 5%, 4%, 3%, 2%, or 1% loss of antigen binding when stored at 40° C. for four weeks, compared to the antibody stored at 5° C. for six months under otherwise substantially identical conditions, such conditions including, e.g., the pHs described above and/or the antibody/ADC concentrations, buffer components, sugar components and/or surfactant components described in the exemplary formulations below.

A therapeutic protein (e.g., an antibody or ADC as described herein) may be present in a formulation at a concentration, e.g., from 1 mg/ml to 200 mg/ml, and in particular embodiments, from 5 to 50 mg/ml, and in particular embodiments at 1 mg/ml, 2 mg/ml, 3 mg/ml, 4 mg/ml, 5 mg/ml, 10 mg/ml, 15 mg/ml, 20 mg/ml, 25 mg/ml, 30 mg/ml, 40 mg/ml, 50 mg/ml, 60 mg/ml, 70 mg/ml, 80 mg/ml, 90 mg/ml or 100 mg/ml. In various embodiments, the concentration of the therapeutic protein is suitable for administration to a subject and provides a therapeutic effect upon administration to the subject. In a particular embodiment, an anti-STEAP-1 antibody or ADC is at a concentration of 1 mg/ml, 2 mg/ml, 3 mg/ml, 4 mg/ml, 5 mg/ml, 10 mg/ml, 15 mg/ml, 20 mg/ml, or 25 mg/ml.

In another aspect, a formulation comprises a histidine-acetate buffer, e.g., at a pH as provided above. Histidine acetate may be at a concentration from 1 mm to 100 mM, and in certain embodiments, at 5, 10, 15, 20, 25, 30, or 40 mM. Histidine acetate buffers are described, e.g., in WO 2006/044908, which is expressly incorporated herein by reference. In an exemplary embodiment, a histidine acetate buffer is used for a “naked” antibody, e.g., a naked anti-STEAP-1 antibody, or alternatively, for an ADC, e.g., an anti-STEAP-1 ADC. In another aspect, a formulation comprises a histidine chloride buffer. Histidine chloride may be at a concentration from 1 mm to 100 mM, and in certain embodiments, at 5, 10, 15, 20, 25, 30, or 40 mM. In an exemplary embodiment, a histidine chloride buffer is used for an ADC, e.g., an anti-STEAP-1 ADC, or alternatively, for a “naked” antibody, e.g., a naked anti-STEAP-1 antibody. In a further exemplary embodiment, a histidine chloride buffer is used when the formulation is to be lyophilized.

In another aspect, a formulation comprises a saccharide. In one such embodiment, the saccharide is selected from the group consisting of trehalose and sucrose. In one such embodiment, trehalose or sucrose is present in an amount from about 60 mM to about 250 mM. In specific embodiments, trehalose or sucrose is present at 100 mM, 125 mM, 150 mM, 175 mM, 200 mM, 210 mM, 220 mM, 230 mM, 240 mM, or 250 mM.

In another aspect, a formulation comprises a surfactant. In one such embodiment, the surfactant is polysorbate 20 (commercially known as TWEEN 20). In one such embodiment, the polysorbate 20 is present at a concentration from about 0.005% to about 0.1%. In specific embodiments, polysorbate 20 is present at a concentration of 0.005%, 0.01%, 0.0125%, 0.015%, 0.0175%, 0.02%, 0.025%, 0.03%, 0.04%, 0.05%, 0.06%, 0.07%, 0.08%, 0.09% or 0.1% polysorbate 20.

In another aspect, a formulation at a pH provided above comprises one or more of a histidine-acetate buffer, a saccharide, and a surfactant, as in any of the embodiments provided above. In a further aspect, a formulation at a pH provided above comprises one or more of a histidine-chloride buffer, a saccharide, and a surfactant, as in any of the embodiments provided above.

### IV. Treatment with Formulations

In one embodiment, the invention provides a method of treating a disease or disorder in a subject comprising administering a formulation described herein to a subject in an amount effective to treat the disease or disorder.

Where a formulation comprises an anti-STEAP-1 antibody (including “naked” anti-STEAP-1 antibodies as well as ADCs), the formulation can be used to treat cancer. The cancer will generally comprise STEAP-1-expressing cells, such that the anti-STEAP-1 antibody is able to bind to the cancer cells. Thus, an invention in this embodiment concerns a method for treating STEAP-1-expressing cancer in a subject, the method comprising administering to the subject a formulation comprising an anti-STEAP-1 antibody as described herein in an amount effective to treat the cancer. Various cancers that can be treated with such a formulation include prostate cancer, Ewing's sarcoma, lung cancer, colon cancer, bladder cancer, ovarian cancer, and pancreatic cancer. See Hubert et al., Proc. Natl. Acad. Sci. USA 96:14523-14528 (1999); WO 99/62941; Challita-Eid et al. Cancer Res. 67:5798-5805; and WO2008/052187.

A patient may be treated with a combination of the antibody formulation, and a chemotherapeutic agent. The combined administration includes coadministration or concurrent administration, using separate formulations or a single formulation, and consecutive administration in either order. Thus, the chemotherapeutic agent may be administered prior to, or following, administration of the antibody formulation. In this embodiment, the timing between at least one administration of the chemotherapeutic agent and at least one administration of the antibody formulation is preferably approximately 1 month or less, and most preferably approximately 2 weeks or less. Alternatively, the chemotherapeutic agent and the antibody formulation are administered concurrently to the patient, in a single formulation or separate formulations.

A patient may be treated with a combination of an anti-STEAP-1 antibody formulation, and a second antibody. The second antibody may comprise an antibody that binds to a prostate cell surface antigen, e.g., Annexin 2, Cadherin-1, Cav-1, Cd34, CD44, EGFR, EphA2, ERGL, Fas, hepsin, HER2, KAI1, MSR1, PATE, PMEPA-1, Prostasin, Prostein, PSCA, PSGR, PSMA, RTVP-1, ST7, TMPRSS2, TRPM2, and Trp-p8. The combined administration includes coadministration or concurrent administration, using separate formulations or a single formulation, and consecutive administration in either order. Thus, the second antibody may be administered prior to, or following, administration of the anti-STEAP-1 antibody formulation. In this embodiment, the timing between at least one administration of the second antibody and at least one administration of the anti-STEAP-1 antibody formulation is preferably approximately 1 month or less, and most preferably approximately 2 weeks or less. Alternatively, the anti-STEAP-1 antibody formulation and the second antibody are administered concurrently to the patient, in a single formulation or separate formulations.

Treatment with a formulation as described herein will preferably result in an improvement in the signs or symptoms of cancer. For instance, such therapy may result in an improvement in survival (overall survival and/or progression free survival) and/or may result in an objective clinical response (partial or complete). Moreover, treatment with the combination of the chemotherapeutic agent and the antibody formulation may result in a synergistic, or greater than additive, therapeutic benefit to the patient.

A formulation can be administered to a human patient in accord with known methods, such as intravenous administration, e.g., as a bolus or by continuous infusion over a period of time, by intramuscular, intraperitoneal, intracerobrospinal, subcutaneous, intra-articular, intrasynovial, intrathecal, oral, topical, or inhalation routes. Intravenous, intramuscular or subcutaneous administration of antibody composition is preferred, with intravenous administration being most preferred.

For subcutaneous delivery, the formulation may be administered via syringe; injection device (e.g. the INJECT-EASE™ and GENJECT™ device); injector pen (such as the GENPEN™); needleless device (e.g. MEDIJECTOR™ and BIOJECTOR™); or subcutaneous patch delivery system.

For the prevention or treatment of disease, the appropriate dosage of the antibody will depend on the type of disease to be treated, as defined above, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 50 mg/kg (e.g. 0.1-20 mg/kg) of anti-STEAP-1 antibody is an initial candidate dosage for administration to the patient, whether, for example, by one or more separate administrations, or by continuous infusion. The dosage of the antibody will generally be in the range from about 0.05 mg/kg to about 10 mg/kg. If a chemotherapeutic agent is administered, it is usually administered at dosages known therefor, or optionally lowered due to combined action of the drugs or negative side effects attributable to administration of the chemotherapeutic agent. Preparation and dosing schedules for such chemotherapeutic agents may be used according to manufacturers' instructions or as determined empirically by the skilled practitioner. Preparation and dosing schedules for such chemotherapy are also described in Chemotherapy Service Ed., M. C. Perry, Williams & Wilkins, Baltimore, Md. (1992).

Other therapeutic regimens may be combined with the antibody including, but not limited to: a second (third, fourth, etc) chemotherapeutic agent(s) (i.e. “cocktails” of different chemotherapeutic agents); another monoclonal antibody; a growth inhibitory agent; a cytotoxic agent; a chemotherapeutic agent; EGFR-targeted drug; tyrosine kinase inhibitor; anti-angiogenic agent; and/or cytokine; etc. In addition to the above therapeutic regimes, the patient may be subjected to surgical removal of cancer cells and/or radiation therapy.

Formulations as provided herein (e.g., an anti-STEAP-1 antibody formulation) may also be administered for diagnostic purposes, e.g., for in vivo diagnostic imaging. In such embodiments, the antibody may be labeled directly or indirectly for detection.

### V. Articles of Manufacture

In another embodiment of the invention, an article of manufacture is provided which contains a formulation of the present invention and provides instructions for its use. The article of manufacture comprises a container. Suitable containers include, for example, bottles, vials (e.g. dual chamber vials), syringes (such as dual chamber syringes) and test tubes. The container may be formed from a variety of materials such as glass or plastic. The container holds the formulation and the label on, or associated with, the container may indicate directions for use. The container holding the formulation may be a multi-use vial, which allows for repeat administrations (e.g. from 2-6 administrations) of the reconstituted formulation. The article of manufacture may further include other materials desirable from a commercial and user standpoint, including other buffers, diluents, filters, needles, syringes, and package inserts with instructions for use as noted in the previous section.

The invention will be more fully understood by reference to the following examples. They should not, however, be construed as limiting the scope of the invention. All literature and patent citations are incorporated herein by reference.

### EXAMPLES

### A. Identification of Succinimide Intermediate by Ion Exchange Chromatography

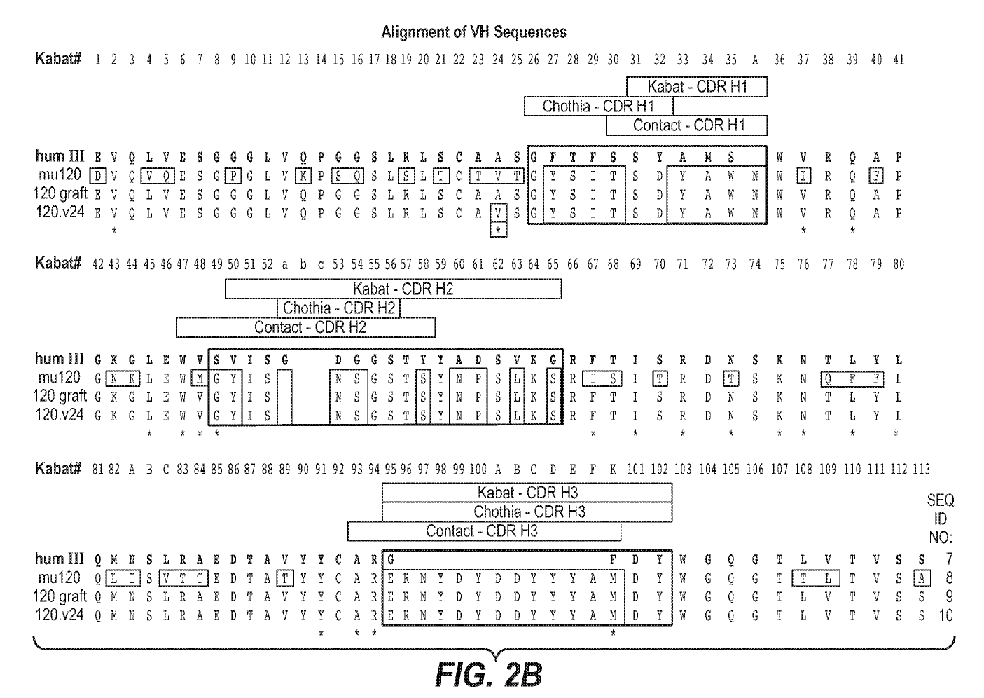

Full length anti-STEAP-1 antibody having heavy and light chain variable regions as in SEQ ID NO:6 and 10, respectively was formulated in 20 mM histidine acetate buffer with 100 mM trehalose and 0.01% Tween 20 at pH 5.5. Samples were kept at 40° C. (“stress conditions”) and analyzed by ion exchange chromatography after 0, 1, 2, or 4 weeks. FIG. 3 shows the resulting elution profile at those time points. A “basic” peak (arrow) increased from 3.9% to 20.7% of the elution peaks with increased time under stress conditions.

The “potency” of the samples was determined by assessing the ability of the antibody to bind antigen in a cell-based assay. In that assay, LB50 cells, which are human embyonic kidney (HEK) 293 cells stably transfected with STEAP-1, were grown in growth medium containing HAM's F12/DMEM (1:1 ratio), 10% FBS with 0.2 mg/mL G418, and 1× GLUTAMAX™ medium (Invitrogen, Carlsbad, Calif.). The STEAP-1 expression level on the cells was determined by Scatchard analysis to be 270,000 sites/cell. The LB50 cells were seeded in a poly-D-Lysine coated 96-well microtiter cell culture plate at 1×10⁵ cells/well and incubated overnight at 37° C. and 5% CO₂. Following incubation, dilutions of anti-STEAP-1 antibody and control samples were prepared in assay diluent (PBS+0.25% BSA) and added to the plate. The plate was then incubated to allow binding of anti-STEAP-1 antibody to STEAP-1 expressed on the LB50 cells. The plate was then washed to remove unbound antibody. Bound anti-STEAP-1 antibody was detected with anti-human IgG-horseradish peroxidase (HRP) and SureBlue Reserve™ tetramethylbenzidine peroxidase (TMB) substrate solution, which produces a colorimetric signal proportional to the amount of bound anti STEAP-1 antibody. As shown in the last column of the table in FIG. 3, increased time under stress conditions resulted in increased loss of potency of the anti-STEAP-1 antibody.

Fractions corresponding to the ion exchange peaks were collected, and mass spectrometry was performed. The basic peak (arrow in FIG. 3) had a mass of 18 Da less than the main peak, indicating a succinimide intermediate. The presence of a succinimide intermediate suggested the presence of either deamidation of asparagine or isomerization of aspartic acid.

### B. Identification of Iso-Asp by Peptide Mapping and ETD-MS

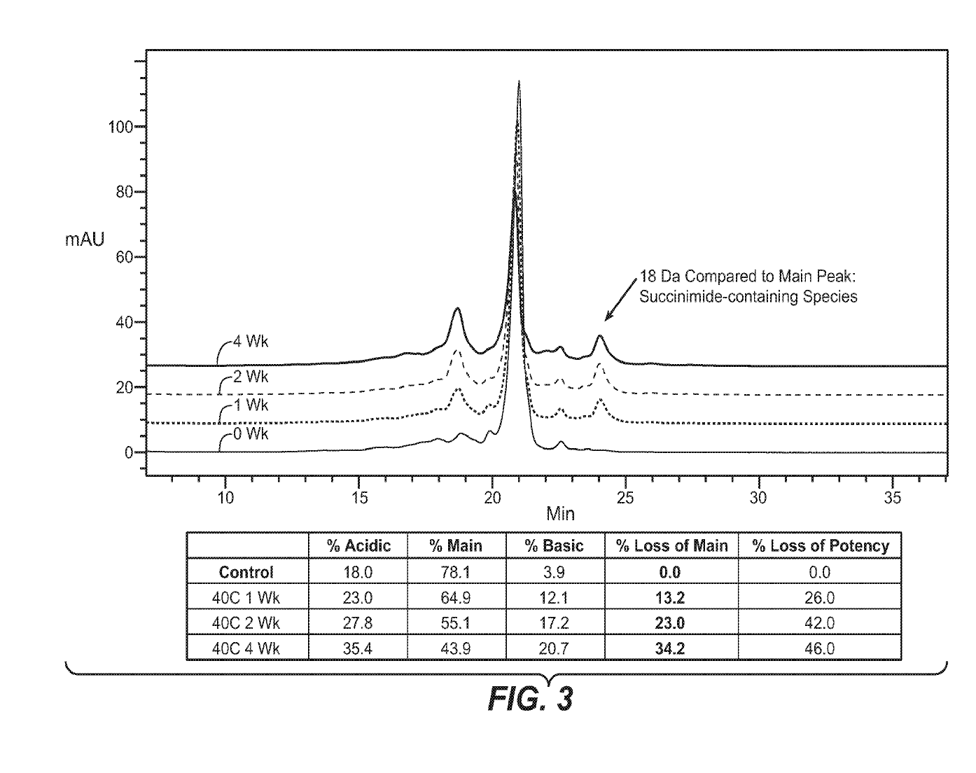

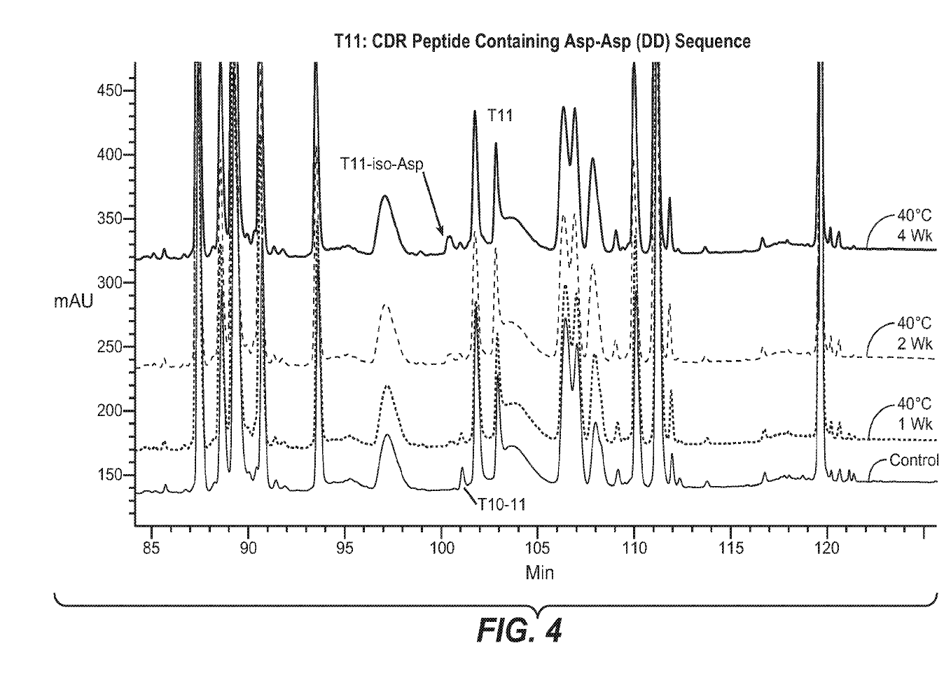

Peptide (tryptic) mapping was performed on the samples. As shown in FIG. 4, two peptides (T11 and T11-iso-Asp) ran differently by reverse phase chromatography, indicating that the two peptides presented different charged surfaces. However, the two peptides had the same mass as determined by mass spectrometry, suggesting that one of the peptides contained iso-Asp. Electron transfer dissociation was used to fragment the peptides, yielding data showing that the first Asp in the Asp-Asp sequence of HVR-H3 (CDR3) was isomerizing, as shown in FIG. 5. That Asp corresponds to position 5 of the peptide shown in FIG. 5 (NYDYDDYYYAMDYWGQGTLVTVSSCSTK (SEQ ID NO:17)), which corresponds to position 7 of SEQ ID NO:16 above.

### C. Effect of Increased pH

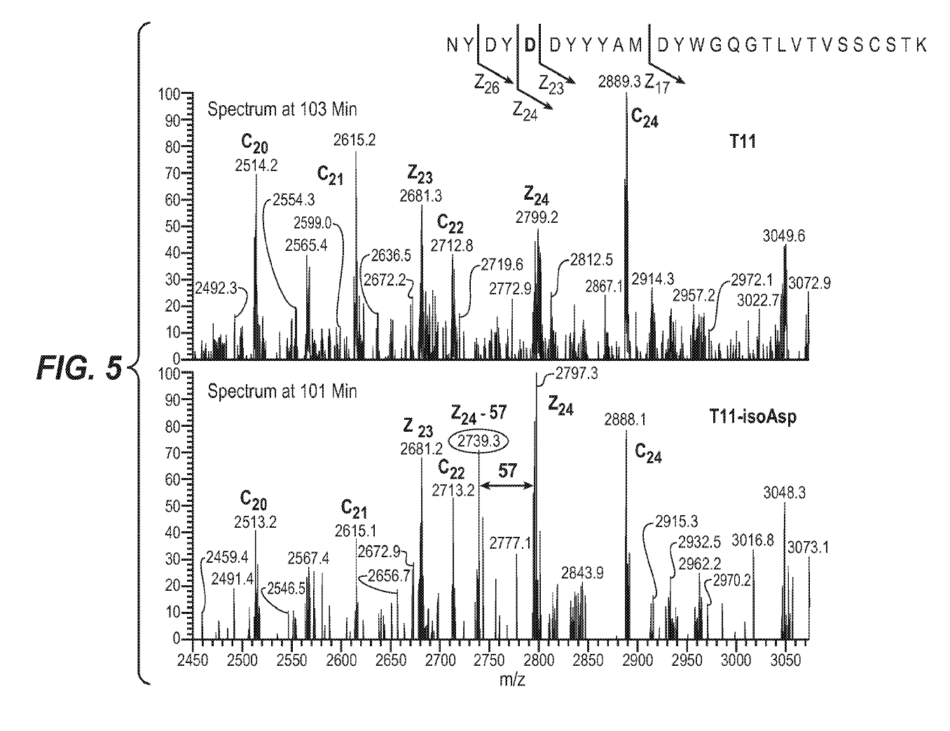

The anti-STEAP-1 antibody of Example A was formulated in 20 mM histidine chloride buffer, 240 mM sucrose and 0.02% Tween 20 at various pHs, as indicated in FIG. 6. When stored for 4 weeks at 40° C. and pH 5.5, the antibody showed loss of binding to STEAP-1 antigen. Formulations with increased pH showed decreased loss of binding at 40° C. No loss of binding was observed at any of the pHs tested when the formulations were stored at 5° C. for six months.

### D. HIC Detection of Iso-Asp and Succinimide

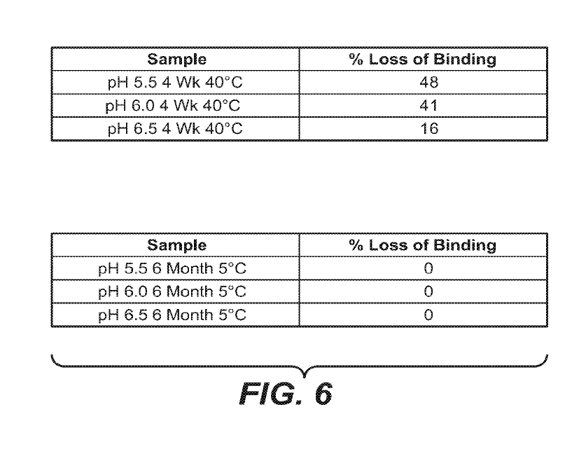

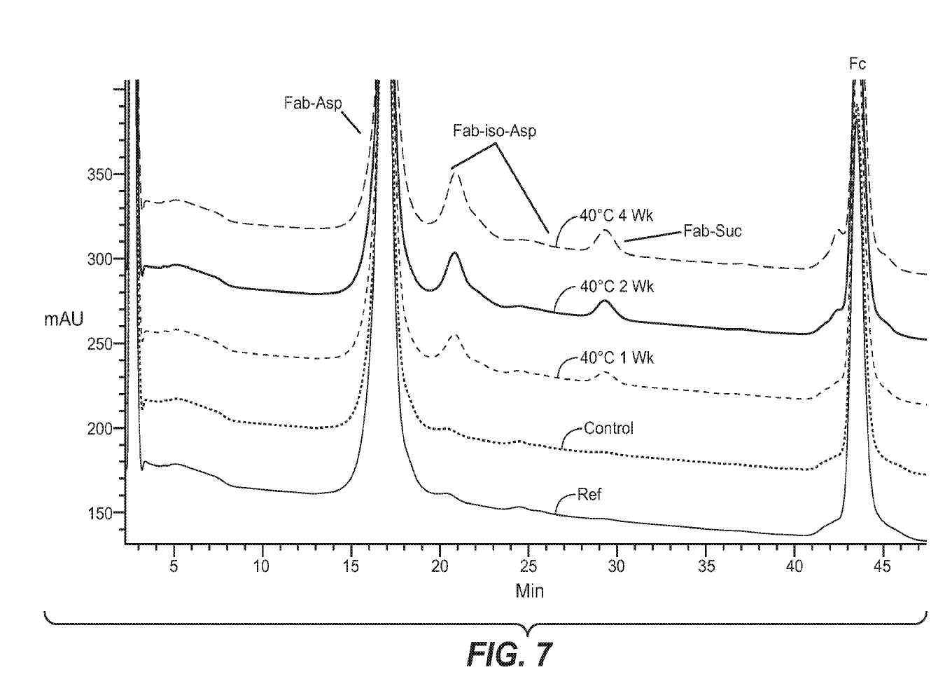

Hydrophobic interaction chromatography (HIC) was used to quantify the amount of iso-Asp and succinimide in anti-STEAP-1 antibody formulated as described above in Example C at pH 5.5 and stored at 40° C. for 0, 1, 2, and 4 weeks. FIG. 7 shows elution profiles containing iso-Asp and succinimide, as indicated. FIG. 8 shows the amount (expressed as a percentage) of iso-Asp and succinimide in the anti-STEAP-1 antibody stored at various temperatures and for various time periods, as indicated. HIC was needed to quantify the amount of iso-Asp and succinimide because the iso-Asp peak appeared under the main peak using ion exchange chromatography.

### E. Rates of Asp to Iso-Asp Isomerization

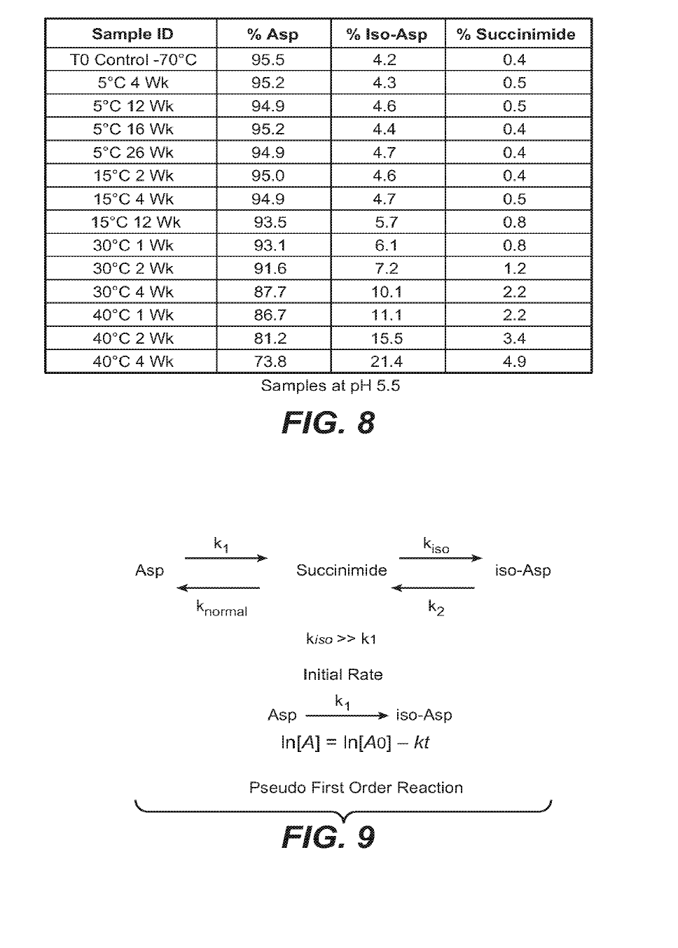

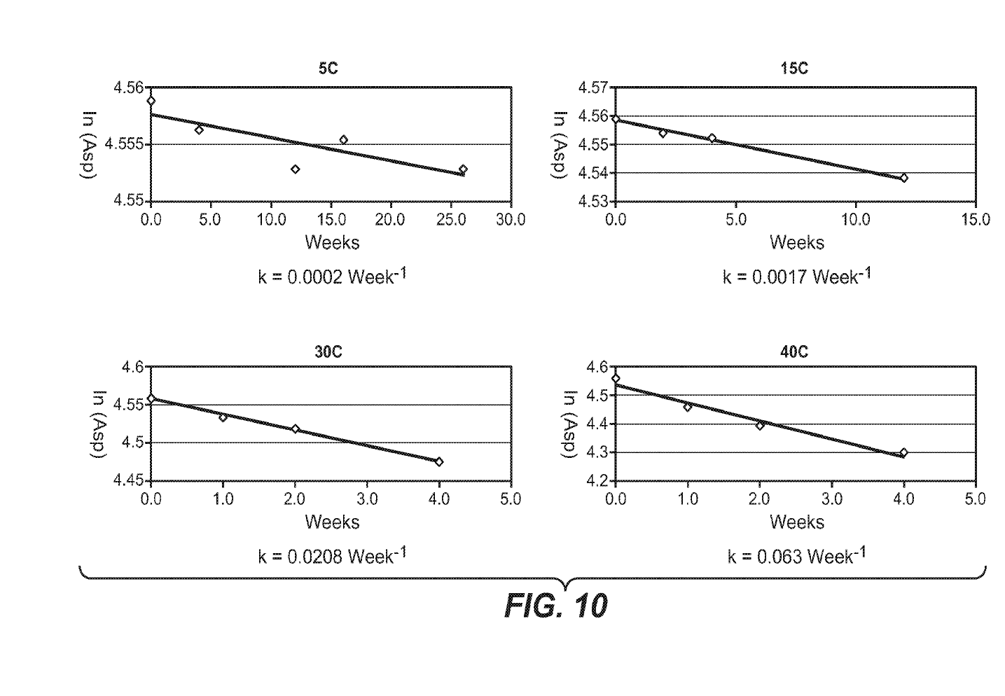

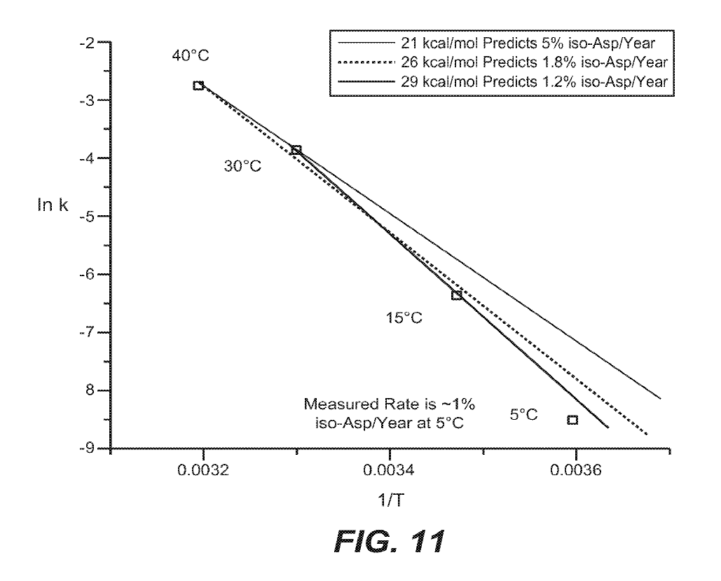

Anti-STEAP-1 antibody was formulated as described above in Example C at pH 5.5. Assuming first order kinetics for the reaction of Asp to iso-Asp (FIG. 9), the rates of Asp to iso-Asp isomerization was determined at various temperatures (FIG. 10). An Arrhenius plot (FIG. 11) was generated using the rates determined in FIG. 10. The plot predicts an activation energy of Asp-Asp isomerization to be about 25-30 kcal/mol.

Although the foregoing invention has been described in some detail by way of illustration and example for purposes of clarity of understanding, the descriptions and examples should not be construed as limiting the scope of the invention. The disclosures of all patent and scientific literatures cited herein are expressly incorporated in their entirety by reference.

What is claimed is:

1. A formulation comprising a therapeutic protein having an Asp-Asp motif, wherein the formulation has a pH that inhibits aspartyl isomerization of an Asp residue in the Asp-Asp motif.

2. A formulation comprising a therapeutic protein having an Asp-Asp motif, wherein the pH of the formulation is greater than 6.0 and less than 9.0.

3. The formulation of claim 2, wherein the pH is from 6.25 to 7.0.

4. The formulation of claim 3, wherein the pH is about 6.5.

5. The formulation of claim 2, wherein the therapeutic protein is an antibody.

6. The formulation of claim 5, wherein the antibody comprises a hypervariable region (HVR) that comprises an Asp-Asp motif.

7. The formulation of claim 6, wherein the Asp-Asp motif occurs in HVR-H3.

8. The formulation of claim 7, wherein the antibody is an anti-STEAP-1 antibody that comprises an HVR-H3 comprising the amino acid sequence of SEQ ID NO:16.

9. The formulation of claim 8, wherein the anti-STEAP-1 antibody further comprises one or more HVRs selected from (a) an HVR-H1 comprising the amino acid sequence of SEQ ID NO:14; (b) an HVR-H2 comprising the amino acid sequence of SEQ ID NO:15; (c) an HVR-L1 comprising the amino acid sequence of SEQ ID NO:11; (d) an HVR-L2 comprising the amino acid sequence of SEQ ID NO:12; and (e) an HVR-L3 comprising the amino acid sequence of SEQ ID NO:13.

10. The formulation of claim 9, wherein the antibody comprises (a) an HVR-H1 comprising the amino acid sequence of SEQ ID NO:14; (b) an HVR-H2 comprising the amino acid sequence of SEQ ID NO:15; (c) an HVR-H3 comprising the amino acid sequence of SEQ ID NO:16; (d) an HVR-L1 comprising the amino acid sequence of SEQ ID NO:11; (e) an HVR-L2 comprising the amino acid sequence of SEQ ID NO:12; and (f) an HVR-L3 comprising the amino acid sequence of SEQ ID NO:13.

11. The formulation of claim 8, wherein the antibody comprises a heavy chain variable region (VH), wherein the VH comprises an amino acid sequence having at least 90% amino acid sequence identity to an amino acid sequence selected from SEQ ID NOs:8-10.

12. The formulation of claim 11, wherein the antibody further comprises a light chain variable region (VL), wherein the VL comprises an amino acid sequence having at least 90% amino acid sequence identity to an amino acid sequence selected from SEQ ID NOs:5-6.

13. The formulation of claim 8, wherein the antibody is conjugated to a cytotoxic agent.

14. The formulation of claim 13, wherein the cytotoxic agent is an auristatin.

15. The formulation of claim 13, wherein the cytotoxic agent is a maytansinoid drug moiety.

16. The formulation of any one of claims 5-8, wherein the antibody shows ≦25% loss of antigen binding when stored at 40° C. for four weeks, compared to storage at 5° C. for six months.

17. The formulation of any one of claims 5-8 comprising a histidine-acetate buffer at a concentration of 20 mM.

18. The formulation of any one of claims 5-8 comprising a histidine-chloride buffer at a concentration of 20 mM.

19. The formulation of any one of claims 5-8 comprising a saccharide selected from trehalose and sucrose present in an amount from 60 mM to 250 mM.

20. The formulation of any one of claims 5-8 comprising polysorbate 20 in an amount from 0.01% to 0.1%.

21. A method of treating cancer, the method comprising administering to a mammal a formulation as in claim 8.

22. A method of inhibiting aspartyl isomerization in a therapeutic protein comprising an Asp-Asp motif, wherein the therapeutic protein is contained in a formulation, the method comprising raising the pH of the formulation to a pH sufficient to inhibit aspartyl isomerization.

23. The method of claim 22, wherein the therapeutic protein is an antibody.
